(19) United States
(12) Patent Application Publication
(10) Pub. No.: US 2015/0299250 A1
(43) Pub. Date: Oct. 22, 2015

(54) MACROCYCLIC COMPOUNDS AND USES THEREOF

(71) Applicant: ADELAIDE RESEARCH & INNOVATION PTY LTD, Adelaide, South Australia (AU)

(72) Inventors: Andrew David Abell, Dulwich, South Australia (AU); Krystle Chua, Singapore (SG); Markus Pietsch, bONN (DE)

(21) Appl. No.: 14/443,220

(22) PCT Filed: Nov. 15, 2013
(86) PCT No.: PCT/AU2013/001322

§ 371 Date: May 15, 2015

(30) Foreign Application Priority Data
Nov. 16, 2012 (AU) 2012904986

(51) Int. Cl. C07K 5/068; C12Q 1/37

(52) U.S. Cl. CPC C07K 5/06086; C12Q 1/37; G01N 2500/04; G01N 2500/10

(57) ABSTRACT

The present invention relates to novel macrocyclic compounds of Formula I and their use as novel therapeutic agents for example as novel compounds used in methods of preventing and/or treating a disease, condition or state in a subject associated with dysregulation of protease activity and/or dysregulation of proteosome activity

### FIELD

The present disclosure relates to macrocyclic compounds, uses of macrocyclic compounds, and screening of macrocyclic compounds.

### BACKGROUND

Proteases are group of enzymes whose catalytic function is to hydrolyze peptide bonds in proteins and which are typically divided into into six broad groups: serine proteases, threonine proteases, cysteine proteases, aspartate proteases and metalloproteases.

Proteases regulate most physiological processes in some way by controlling the activation, synthesis and turnover of proteins. Dysregulation of protease activity can also lead to a variety of diseases or undesired physiological conditions or states. As such proteases represent important potential targets for medical intervention because of their important regulatory roles in these processes.

For example, Calpains are Ca²⁺-dependent cysteine proteases that are involved in the regulation of wide variety of biological processes. An elevation in calpain activity has been implicated in a number of medical conditions, including traumatic brain injury, muscular dystrophy and cataracts. In the case of cataract formation, elevated levels of calcium in lens and subsequent over-activation of calpain have been linked to the break down of lens protein, resulting in lens opacity and ultimately, blindness.

Compounds that mimic the natural substrates of proteases have been investigated as potential therapeutic targets. However, such compounds have a number of disadvantages, and thus limited therapeutic potential. Some of these disadvantages include one or more of poor stability, low cell permeability, low solubility, low activity, poor selectivity, or high cell toxicity.

Accordingly, there is a need to develop compounds that can be used to regulate protease activity and which address one or more problems in the art and/or provide one or more advantages.

### SUMMARY

The present disclosure relates to macrocyclic compounds, uses of macrocyclic compounds, and screening of macrocyclic compounds for new therapeutic agents.

Certain embodiments of the present disclosure provide a compound of Formula I and/or a pharmaceutically acceptable salt, solvate, tautomer, hydrate or prodrug derivative thereof:

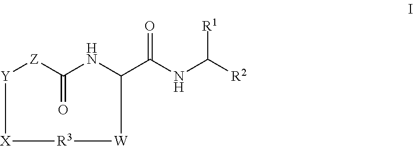

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

Certain embodiments of the present disclosure provide a compound of one of the following formulas and/or a pharmaceutically acceptable salt, solvate, tautomer, hydrate or prodrug derivative thereof:

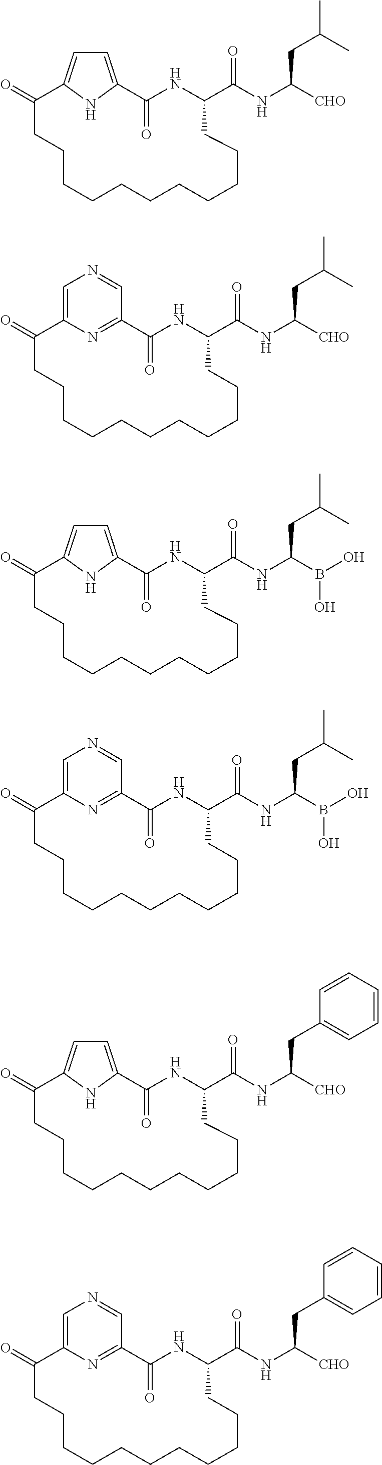 [SMILES: CC(C)C[C@@H](C=O)NC(=O)[C@@H]1CCCCCCCCCCCC(=O)c2ccc([nH]2)C(=O)N1 ; CC(C)C[C@@H](C=O)NC(=O)[C@@H]1CCCCCCCCCCCC(=O)c2cncc(n2)C(=O)N1 ; CC(C)C[C@H](NC(=O)[C@@H]1CCCCCCCCCCCC(=O)c2ccc([nH]2)C(=O)N1)B(O)O ; CC(C)C[C@H](NC(=O)[C@@H]1CCCCCCCCCCCC(=O)c2cncc(n2)C(=O)N1)B(O)O ; O=C[C@H](Cc1ccccc1)NC(=O)[C@@H]1CCCCCCCCCCCC(=O)c2ccc([nH]2)C(=O)N1 ; O=C[C@H](Cc1ccccc1)NC(=O)[C@@H]1CCCCCCCCCCCC(=O)c2cncc(n2)C(=O)N1]

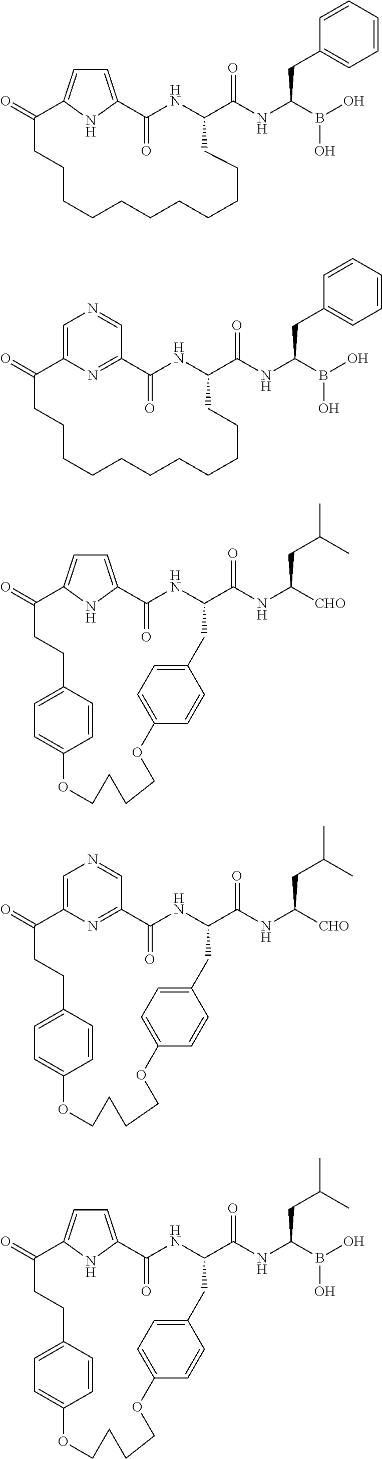 [SMILES: O=C1CCCCCCCCCCC[C@@H](C(=O)N[C@@H](Cc2ccccc2)B(O)O)NC(=O)c2ccc1[nH]2 ; O=C1CCCCCCCCCCC[C@@H](C(=O)N[C@@H](Cc2ccccc2)B(O)O)NC(=O)c2cncc1n2 ; CC(C)C[C@@H](C=O)NC(=O)[C@@H]1Cc2ccc(cc2)OCCCCOc2ccc(cc2)CCC(=O)c2ccc([nH]2)C(=O)N1 ; CC(C)C[C@@H](C=O)NC(=O)[C@@H]1Cc2ccc(cc2)OCCCCOc2ccc(cc2)CCC(=O)c2cncc(n2)C(=O)N1 ; CC(C)C[C@H](NC(=O)[C@@H]1Cc2ccc(cc2)OCCCCOc2ccc(cc2)CCC(=O)c2ccc([nH]2)C(=O)N1)B(O)O]

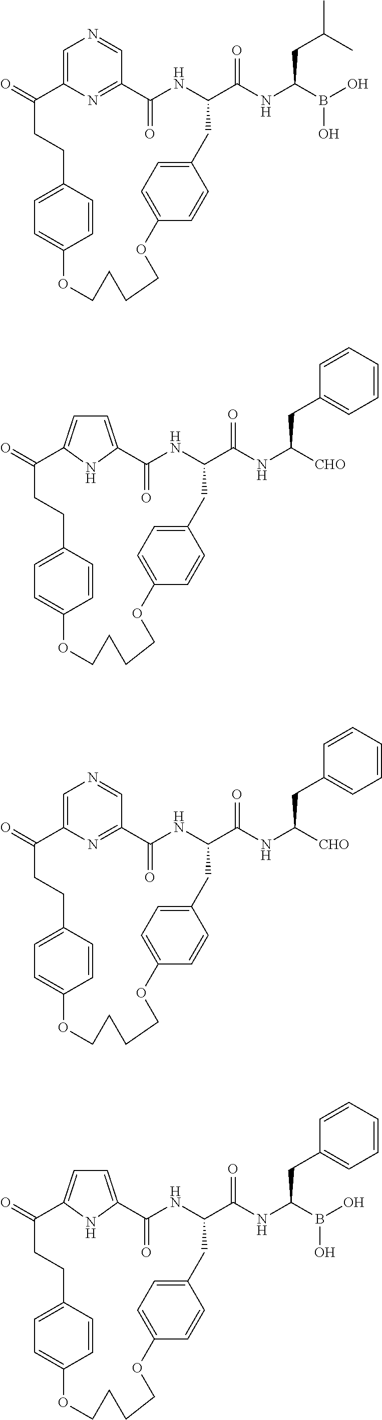 [SMILES: CC(C)C[C@H](NC(=O)[C@@H]1Cc2ccc(cc2)OCCCCOc2ccc(cc2)CCC(=O)c2cncc(n2)C(=O)N1)B(O)O ; O=C[C@H](Cc1ccccc1)NC(=O)[C@@H]1Cc2ccc(cc2)OCCCCOc2ccc(cc2)CCC(=O)c2ccc([nH]2)C(=O)N1 ; O=C[C@H](Cc1ccccc1)NC(=O)[C@@H]1Cc2ccc(cc2)OCCCCOc2ccc(cc2)CCC(=O)c2cncc(n2)C(=O)N1 ; O=C1CCc2ccc(cc2)OCCCCOc2ccc(cc2)C[C@@H](C(=O)N[C@@H](Cc2ccccc2)B(O)O)NC(=O)c2ccc1[nH]2]

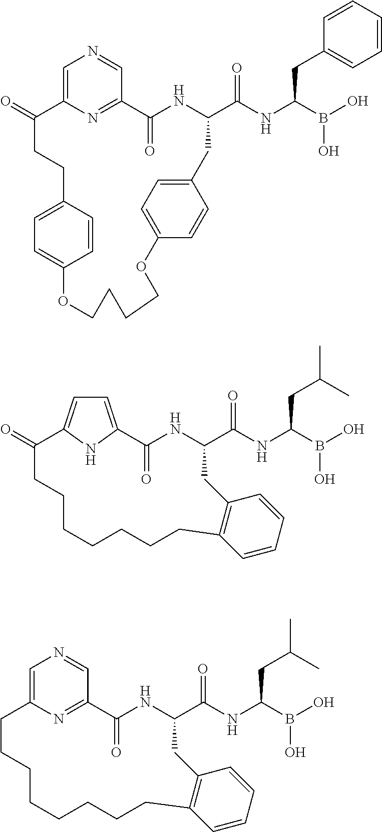 [SMILES: O=C1CCc2ccc(cc2)OCCCCOc2ccc(cc2)C[C@@H](C(=O)N[C@@H](Cc2ccccc2)B(O)O)NC(=O)c2cncc1n2 ; CC(C)C[C@H](NC(=O)[C@@H]1Cc2ccccc2CCCCCCCC(=O)c2ccc([nH]2)C(=O)N1)B(O)O ; CC(C)C[C@H](NC(=O)[C@@H]1Cc2ccccc2CCCCCCCCc2cncc(n2)C(=O)N1)B(O)O]

Certain embodiments of the present disclosure provide a method of preventing and/or treating a disease, condition or state in a subject associated with dysregulation of protease activity and/or dysregulation of proteosome activity, the method comprising administering to the subject a therapeutically effective dose of a compound of Formula I and/or a pharmaceutically acceptable salt, solvate, tautomer, hydrate or prodrug derivative thereof:

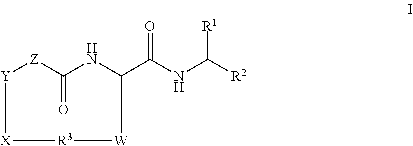

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

Certain embodiments of the present disclosure provide a method of preventing and/or treating an ocular disorder, a cataract, an optic neuropathy, ischemic optic neuropathy, diabetic neuropathy, diabetic macular oedema, glaucoma, macular degeneration, retinal ischaemia, retinal damage, retinal detachment, presbyopia, an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, or a blood-borne cancer, a malignancy, and a multiple myeloma, the method comprising administering to the subject a therapeutically effective dose of a compound of Formula I and/or a pharmaceutically acceptable salt, solvate, tautomer, hydrate or prodrug derivative thereof:

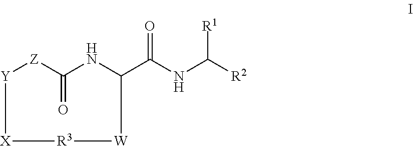

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR4, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

Certain embodiments of the present disclosure provide a method of identifying a protease inhibitor, the method comprising:
(i) providing a P1-P3 or a P2-P4 macrocyclic peptidomimetic compound;
(ii) determining the ability of the P1-P3 or the P2-P4 macrocyclic peptidomimetic compound to inhibit a protease; and
(iii) identifying the P1-P3 or the P2-P4 macrocyclic peptidomimetic compound as a protease inhibitor.

Certain embodiments of the present disclosure provide a method of identifying a therapeutic agent for preventing and/or treating a disease, condition or state associated with dysregulation of protease activity and/or dysregulation of proteosome activity, the method comprising:
(i) providing a P1-P3 or a P2-P4 macrocyclic peptidomimetic compound;
(ii) determining the ability of the P1-P3 or the P2-P4 macrocyclic peptidomimetic compound to prevent and/or treat a disease, condition or state associated with dysregulation of protease activity and/or dysregulation of proteosome activity; and
(iii) identifying the P1-P3 or the P2-P4 macrocyclic peptidomimetic compound as a therapeutic agent for preventing and/or treating a disease, condition or state associated with dysregulation of protease activity and/or dysregulation of proteosome activity.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows the molecular structure and atom labelling scheme of 6a (ORTEP). Displacement ellipsoids are shown at 50% probability label for non-H atoms. H atoms are depicted as small circles of arbitrary radii.

FIG. 2(A) shows cell viability assays of MEFs p53^{+/+} or p53^{-/-} exposed to Bortezomib, MG132 or compounds 1 to 6. Dose-response curves are provided in Supplementary Figure S3. Compound 1 was not assayed using this system. FIG. 2(B) shows Western blot analysis of p53 protein expression in MEF p53^{+/+} or p53^{-/-} following treatment with MG132 at indicated concentrations for 16 hours. β-tubulin is used as a loading control.

### DETAILED DESCRIPTION

The present disclosure relates to macrocyclic compounds, uses of macrocyclic compounds, and screening of macrocyclic compounds.

Certain embodiments of the present disclosure provide a macrocyclic compound as described herein.

In certain embodiments, the macrocyclic compound comprises a peptide mimetic, also referred to herein as a “peptidomimetic compound”. In certain embodiments, the macrocyclic compound comprises a P1-P3 or a P2-P4 macrocyclic peptidomimetic compound.

Certain embodiments of the present disclosure provide a compound of Formula I and/or a pharmaceutically acceptable salt, solvate, tautomer, hydrate or prodrug derivative thereof:

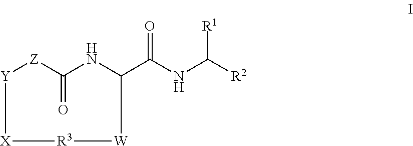

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

As used herein, the term “side chain of a natural or non-natural alpha-amino acid” refers to the group RA in a natural or non-natural amino acid of formula NH₂—CH(RA)-COOH, and/or a derivative thereof.

As used herein, the term “natural alpha-amino acid includes the 20 L-amino acids (or a residue thereof) which comprise most polypeptides in living systems, for example: alanine (Ala); arginine (Arg); asparagine (Asn); aspartic acid (Asp); cysteine (Cys); glutamine (Gln); glutamic acid (Glu); glycine (Gly); histidine (His); isoleucine (Ile); leucine (Leu); lysine (Lys); methionine (Met); phenylalanine (Phe); proline (Pro); serine (Ser); threonine (Thr); tryptophan (Trp); tyrosine (Tyr); and valine (Val). The term also includes rarer amino acids, for example, 4-hydroxyproline, 5-hydroxylysine, N-methyllysine, 3-methylhistidine, desmosine and isodesmosine, and naturally occurring amino acids not found in proteins (for example, gamma-aminobutyric acid, homocysteine, homoserine, citrulline, ornithine, canavanine, djenkolic acid and beta-cyanoalanine). Other natural amino acids are contemplated.

Natural alpha-amino acids which contain functional substituents, for example amino, carboxyl, hydroxy, mercapto, guanidyl, imidazolyl or indolyl groups in their characteristic side chains include arginine, lysine, glutamic acid, aspartic acid, tryptophan, histidine, serine, threonine, tyrosine and cysteine.

As used herein, the term “non-natural alpha-amino acid” includes any alpha-amino acid (or residue thereof) other than the natural amino acids listed above. Non-natural amino acids include the D-isomers of the natural L-amino acids. Non-natural amino acids also include, but are not limited to: D-phenylalanine; norleucine; hydroxyproline; alpha-carboxyglutamic acid; and pyroglutamic acid. Other amino acids are contemplated.

As used herein, the term “pharmaceutically acceptable salt” includes acid addition salts of any basic moiety that may be present in a compound of the present disclosure, and base addition salts of any acidic moiety that may be present in a compound of the present disclosure. Such salts are typically prepared by reacting the compound with a suitable organic or inorganic acid or base. Examples of pharmaceutically acceptable salts of basic moieties include: sulfates; methanesulfonates; acetates; hydrochlorides; hydrobromides; phosphates; toluenesulfonates; citrates; maleates; succinates; tartrates; lactates; and fumarates. Examples of pharmaceutically acceptable salts of acidic moieties include: ammonium salts; alkali metal salts such as sodium salts and potassium salts; and alkaline earth metal salts such as calcium salts and magnesium salts. Other pharmaceutically acceptable salts are contemplated and will be apparent to those skilled in the art.

As used herein, the term “prodrug derivative” includes functional derivatives of the compounds of the present disclosure, the pharmacological action of which results from conversion to a compound of the present disclosure I by metabolic processes within the body. Prodrug derivatives are generally prepared by modifying functional groups in such a. way that the modification is modified in vivo to yield the parent compound. Conventional procedures for the selection and preparation of suitable prodrug derivatives are known to those persons skilled in the art and are discussed in, for example, T. Higuchi and V. Stella, Pro-drugs as Novel Delivery Systems, volume 14 of the A. C. S. Symposium Series, 1987, and E. B. Roche (ed.), Bioreversible Carriers in Drug Design, Pergamon Press, New York, 1987.

The compounds of the present disclosure may be in the form of tautomers, hydrates, or solvates with pharmaceutically acceptable solvents. The present disclosure contemplates such tautomers, hydrates and solvates, as well as the corresponding unsolvated forms.

As used herein, the term “optionally substituted” includes one or more hydrogen atoms in the group indicated is replaced with one or more independently selected suitable substituents, provided that the normal valency of each atom to which the optional substituent/s are attached is not exceeded, and that the substitution results in a stable compound.

Unless a moiety of a compound is defined as being unsubstituted, that moiety may be optionally substituted. In certain embodiments, the optional substituents are independently selected from the group consisting of alkyl, alkoxyalkyl, aminoalkyl, haloalkyl, hydroxyalkyl, alkenyl, alkynyl, alkoxy, haloalkoxy, aryl, arylalkyl, arylalkoxy, aryloxy, heterocyclyl, heterocyclylalkyl, heteroaryl, heteroarylalkoxy, heteroarylalkyl, heteroaryloxy, carboxy, oxo, acyl, amido, nitro, cyano, hydroxyl and halo; —O(C═O)—R^{X}, —C(═O)O—R^{X}, —C(═O)—R^{X}, NH—C(═O)—R^{X}, —S—R^{x}, —S(═O)—R^{X} and S(═O)₂—R^{X}, wherein each R^{x} is independently selected from alkyl, aryl, heterocyclyl and heteroaryl; —NR^{y}R^{z}, -alkyl-NR^{y}R^{z}, —C(═O)—NR^{y}R^{z}, —S(═O)—NR^{y}R^{z} and —S(═O)₂—NR^{y}R^{z}, wherein each R^{y} and R^{z} is independently selected from hydrogen, alkyl, aryl, heterocyclyl and heteroaryl.

The general chemical terms used in the formulae herein have their usual meanings.

In this regard, the term “alkyl” includes straight chain, branched chain or cyclic saturated hydrocarbon groups. In certain embodiments, the alkyl groups comprise 1 to 6 carbon atoms. In certain embodiments, the alkyl group is methyl, ethyl, n-propyl, iso-propyl, cyclopropyl, n-butyl, iso-butyl, tert-butyl or cyclobutyl. Other alkyl groups are contemplated. The alkyl group may be optionally substituted.

The term “alkenyl” includes straight chain, branched chain or cyclic mono-unsaturated hydrocarbon groups. The alkenyl group may be optionally substituted.

The term “alkoxy” includes the groups alkyl-O—.

The term “aryl” includes aromatic radicals including, but not limited to: phenyl; naphthyl; indanyl; biphenyl; and the like. The aryl group may be optionally substituted. In certain embodiments, the aryl group comprises 4 to 10 carbon atoms.

The term “aryloxy” includes the groups aryl-O—.

The term “arylalkoxy” includes the groups aryl-alkyl-O—.

The term “arylalkyl” includes the groups aryl-alkyl-.

The term “heteroaryl” includes heteroaromatic radicals including, but not limited to: pyrimidinyl; pyridyl; pyrrolyl; furyl; oxazolyl; thiophenyl; and the like. The heteroaryl group may be optionally substituted.

The term “heteroaryloxy” includes the groups heteroaryl-O—.

The term “heteroarylalkoxy” includes the groups heteroaryl-alkyl-O.

The term “heteroarylalkyl” includes the groups heteroaryl-alkyl-.

The term “heterocyclyl” includes non-aromatic saturated heterocyclic radicals including, but not limited to: piperidinyl; pyrrolidinyl; piperazinyl; 1,4-dioxanyl; tetrahydrofuranyl; tetrahydrothiophenyl; and the like. The heterocyclyl group may be optionally substituted.

The term “heterocyclylalkyl” includes the groups heterocyclyl-alky-.

The term “thioalkoxy” includes the groups alkyl-S—.

In certain embodiments, Y is C(═O) in the compound of Formula I.

In certain embodiments, R¹ has the following stereochemistry in the compound of Formula I:

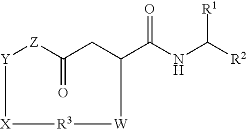

In certain embodiments, R¹ in the compound of Formula I comprises a leucine or a phenylalanine side chain, or a derivative of these side chains. Other side chains are contemplates and are as described herein.

In certain embodiments, R² in the compound of Formula I is B(OH)₂.

In certain embodiments, R² in the compound of Formula I is C(═O)H.

In certain embodiments, W in the compound of Formula I has the following stereochemistry:

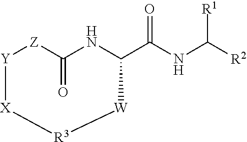

In certain embodiments, where W is no atom, R³ in the compound of Formula I may have the same stereochemistry as W indicated above.

In certain embodiments, in the compound of Formula I, W is no atom, (CH₂), (CHR) or (CR₂), wherein R comprises optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy.

In certain embodiments, in the compound of Formula I, W is no atom or (CH₂).

In certain embodiments, in the compound of Formula I, X is no atom, (CH₂), (CHR) or (CR₂), wherein R comprises optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy.

In certain embodiments, in the compound of Formula I, X is no atom or (CH₂).

In certain embodiments, in the compound of Formula I, X is (CH₂) and W is no atom.

In certain embodiments, in the compound of Formula I, X is no atom and W is (CH₂).

In certain embodiments, in the compound of Formula I, X is no atom and W is no atom.

In certain embodiments, in the compound of Formula I, R³ is (CH₂)₉₋₁₃, (CH₂)₁₁, (CH₂)₂-Phe-O—(CH₂)₄—O-Phe-(CH₂), (CH₂)₆-Phe-(CH₂), (CH₂)₈-Phe-(CH₂), (CH₂)₄-Phe-(CH₂), (CH₂)₅-Phe-(CH₂), (CH₂)₉₋₁₂, (CH₂)₁₀, (CH₂)-Phe-O—(CH₂)₄—O-Phe-(CH₂), (CH₂)₅-Phe-(CH₂), (CH₂)₇-Phe-(CH₂), (CH₂)₃-Phe-(CH₂), (CH₂)₄-Phe-(CH₂), (CH₂)₈₋₁₂, (CH₂)₁₀, (CH₂)₂-Phe-O—(CH₂)₄—O-Phe, (CH₂)₆-Phe, (CH₂)₈-Phe, (CH₂)₄, (CH₂)₅-Phe, (CH₂)₇₋₁₁, (CH₂)₉, (CH₂)-Phe-O—(CH₂)₄—O-Phe, (CH₂)₅-Phe, (CH₂)₇-Phe, (CH₂)₃-Phe, or (CH₂)₄-Phe. The Phe groups may be optionally substituted.

In certain embodiments, in the compound of Formula I, X is no atom, W is no atom and R³ is (CH₂)₉₋₁₃.

In certain embodiments, in the compound of Formula I, X is no atom, W is no atom and R³ is (CH₂)₁₁.

In certain embodiments, in the compound of Formula I, X is no atom, W is no atom and R³ is (CH₂)₂-Phe-O—(CH₂)₄—O-Phe-(CH₂).

In certain embodiments, in the compound of Formula I, X is no atom, W is no atom and R³ is one or (CH₂)₆-Phe-(CH₂), (CH₂)₈-Phe-(CH₂), or (CH₂)₄-Phe-(CH₂).

In certain embodiments, in the compound of Formula I, X is no atom, W is no atom and R³ is (CH₂)₅-Phe-(CH₂).

In certain embodiments, X is (CH₂).

In certain embodiments, in the compound of Formula I, X is (CH₂), W is no atom and R³ is (CH₂)₉₋₁₂.

In certain embodiments, in the compound of Formula I, X is (CH₂), W is no atom and R³ is (CH₂)₁₀.

In certain embodiments, in the compound of Formula I, X is (CH₂), W is no atom and R³ is (CH₂)-Phe-O—(CH₂)₄—O-Phe-(CH₂).

In certain embodiments, in the compound of Formula I, X is no (CH₂), W is no atom and R³ is one or (CH₂)₅-Phe-(CH₂), (CH₂)₇-Phe-(CH₂), or (CH₂)₃-Phe-(CH₂).

In certain embodiments, in the compound of Formula I, X is (CH₂), W is no atom and R³ is (CH₂)₄-Phe-(CH₂).

In certain embodiments, in the compound of Formula I, X is no atom, W is (CH₂) and R³ is (CH₂)₈₋₁₂.

In certain embodiments, in the compound of Formula I, X is no atom, W is (CH₂) and R³ is (CH₂)₁₀.

In certain embodiments, in the compound of Formula I, X is no atom, W is (CH₂) and R³ is (CH₂)₂-Phe-O—(CH₂)₄—O-Phe.

In certain embodiments, in the compound of Formula I, X is no atom, W is (CH₂) and R³ is one or (CH₂)₆-Phe, (CH₂)₈-Phe, or (CH₂)₄.

In certain embodiments, in the compound of Formula I, X is no atom, W is (CH₂) and R³ is (CH₂)₅-Phe.

In certain embodiments, in the compound of Formula I, X is (CH₂), W is (CH₂) and R³ is (CH₂)₇₋₁₁.

In certain embodiments, in the compound of Formula I, X is (CH₂), W is (CH₂) and R³ is (CH₂)₉.

In certain embodiments, in the compound of Formula I, X is (CH₂), W is (CH₂) and R³ is (CH₂)-Phe-O—(CH₂)₄—O-Phe.

In certain embodiments, in the compound of Formula I, X is (CH₂), W is (CH₂) and R³ is one or (CH₂)₅-Phe, (CH₂),-Phe, or (CH₂)₃-Phe.

In certain embodiments, in the compound of Formula I, X is (CH₂), W is (CH₂) and R³ is (CH₂)₄-Phe.

In certain embodiments, in the compound of Formula I, Z is a heterocyclopentyl, a heterocyclohexcyl, a five membered heterocyclic aromatic ring, or a six membered heterocyclic aromatic ring.

In certain embodiments, the compound of Formula I has one of the following formulas:

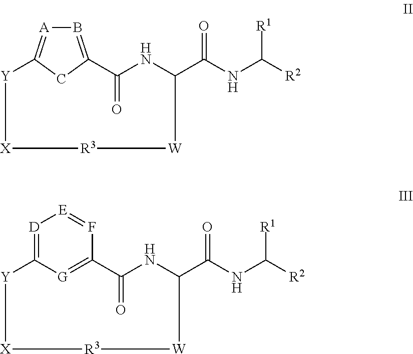

wherein:
R¹, R², Y, X, R³ and W are as previously described herein;
A is O, N, CH or CR⁶, wherein R⁶ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
B is O, N, CH or CR⁷, wherein R⁷ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
C is S, O, NH or NR⁸, wherein R⁸ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
D is N, CH or CR⁹, wherein R⁹ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
E is N, CH or CR¹⁰, wherein R¹⁰ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
F is N, CH or CR¹¹, wherein R¹¹ is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy; and
G is N, CH or CR¹², wherein R¹² is optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy.

In certain embodiments, in the compound of Formula II, A is CH.

In certain embodiments, in the compound of Formula II, B is CH.

In certain embodiments, in the compound of Formula II, C is NH.

In certain embodiments, in the compound of Formula III, D is CH.

In certain embodiments, in the compound of Formula III, E is N.

In certain embodiments, in the compound of Formula III, F is CH.

In certain embodiments, in the compound of Formula III, G is N.

In certain embodiments, in the compound of Formula I, Z is a pyrrole ring.

In certain embodiments, in the compound of Formula I, Z is a pyrazine ring.

In certain embodiments, the compound of Formula I has one of the following formulas and/or a pharmaceutically acceptable salt, solvate, tautomer, hydrate or prodrug derivative thereof:

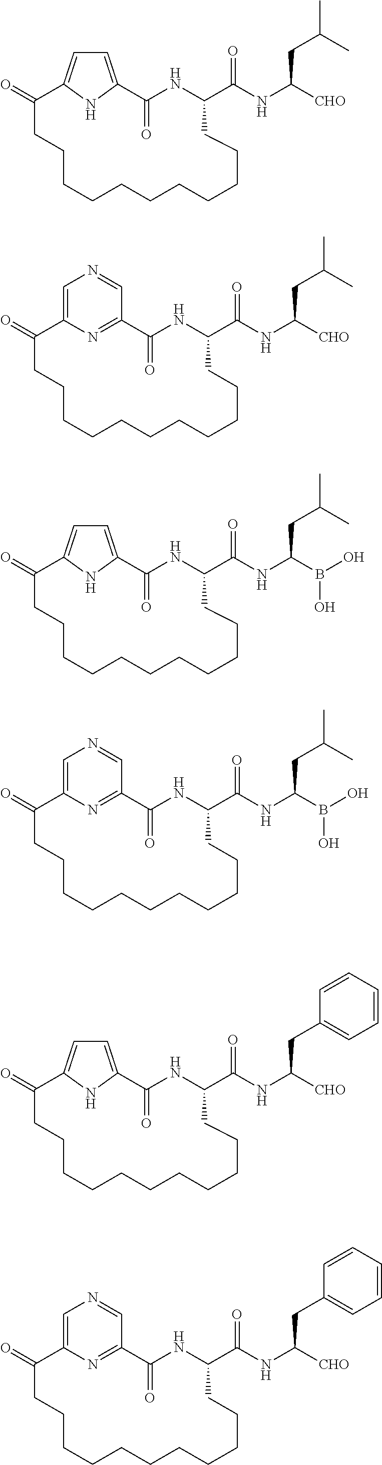 [SMILES: CC(C)C[C@@H](C=O)NC(=O)[C@@H]1CCCCCCCCCCCC(=O)c2ccc([nH]2)C(=O)N1 ; CC(C)C[C@@H](C=O)NC(=O)[C@@H]1CCCCCCCCCCCC(=O)c2cncc(n2)C(=O)N1 ; CC(C)C[C@H](NC(=O)[C@@H]1CCCCCCCCCCCC(=O)c2ccc([nH]2)C(=O)N1)B(O)O ; CC(C)C[C@H](NC(=O)[C@@H]1CCCCCCCCCCCC(=O)c2cncc(n2)C(=O)N1)B(O)O ; O=C[C@H](Cc1ccccc1)NC(=O)[C@@H]1CCCCCCCCCCCC(=O)c2ccc([nH]2)C(=O)N1 ; O=C[C@H](Cc1ccccc1)NC(=O)[C@@H]1CCCCCCCCCCCC(=O)c2cncc(n2)C(=O)N1]

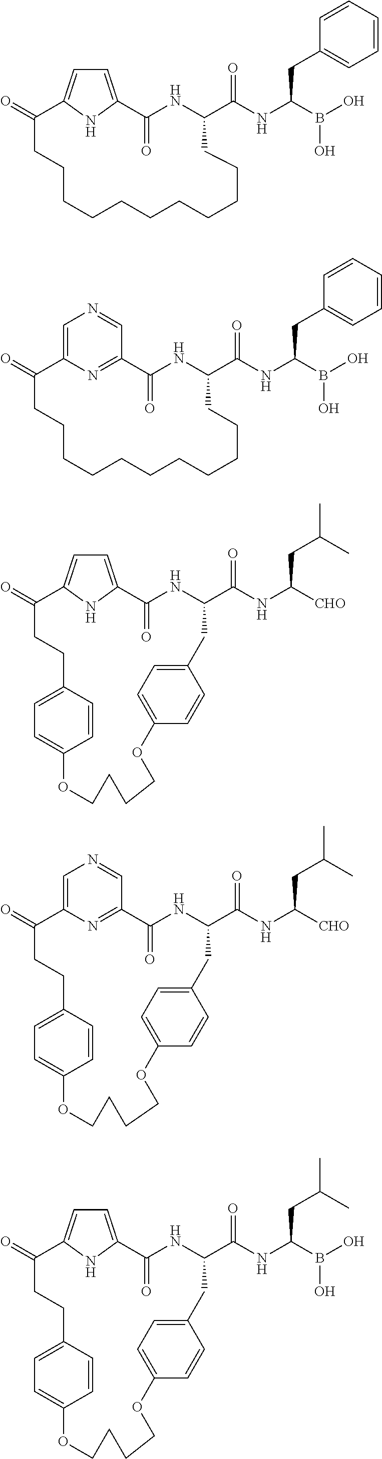 [SMILES: O=C1CCCCCCCCCCC[C@@H](C(=O)N[C@@H](Cc2ccccc2)B(O)O)NC(=O)c2ccc1[nH]2 ; O=C1CCCCCCCCCCC[C@@H](C(=O)N[C@@H](Cc2ccccc2)B(O)O)NC(=O)c2cncc1n2 ; CC(C)C[C@@H](C=O)NC(=O)[C@@H]1Cc2ccc(cc2)OCCCCOc2ccc(cc2)CCC(=O)c2ccc([nH]2)C(=O)N1 ; CC(C)C[C@@H](C=O)NC(=O)[C@@H]1Cc2ccc(cc2)OCCCCOc2ccc(cc2)CCC(=O)c2cncc(n2)C(=O)N1 ; CC(C)C[C@H](NC(=O)[C@@H]1Cc2ccc(cc2)OCCCCOc2ccc(cc2)CCC(=O)c2ccc([nH]2)C(=O)N1)B(O)O]

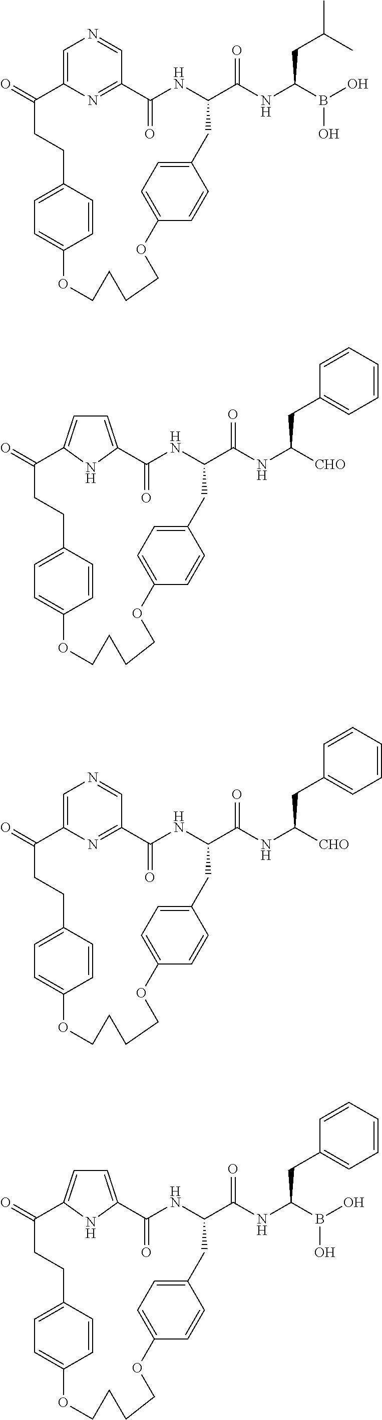 [SMILES: CC(C)C[C@H](NC(=O)[C@@H]1Cc2ccc(cc2)OCCCCOc2ccc(cc2)CCC(=O)c2cncc(n2)C(=O)N1)B(O)O ; O=C[C@H](Cc1ccccc1)NC(=O)[C@@H]1Cc2ccc(cc2)OCCCCOc2ccc(cc2)CCC(=O)c2ccc([nH]2)C(=O)N1 ; O=C[C@H](Cc1ccccc1)NC(=O)[C@@H]1Cc2ccc(cc2)OCCCCOc2ccc(cc2)CCC(=O)c2cncc(n2)C(=O)N1 ; O=C1CCc2ccc(cc2)OCCCCOc2ccc(cc2)C[C@@H](C(=O)N[C@@H](Cc2ccccc2)B(O)O)NC(=O)c2ccc1[nH]2]

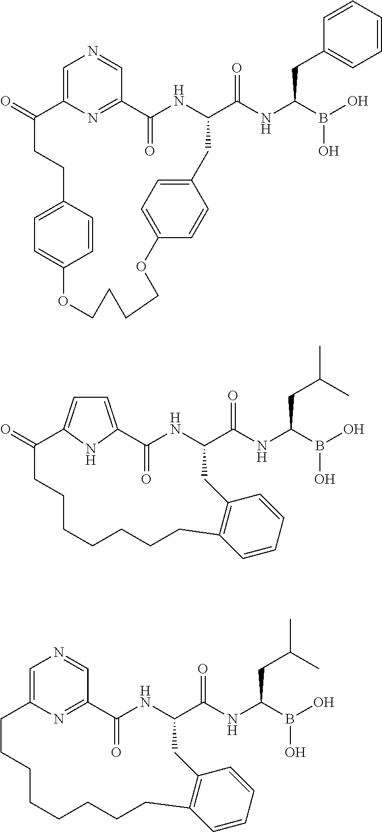 [SMILES: O=C1CCc2ccc(cc2)OCCCCOc2ccc(cc2)C[C@@H](C(=O)N[C@@H](Cc2ccccc2)B(O)O)NC(=O)c2cncc1n2 ; CC(C)C[C@H](NC(=O)[C@@H]1Cc2ccccc2CCCCCCCC(=O)c2ccc([nH]2)C(=O)N1)B(O)O ; CC(C)C[C@H](NC(=O)[C@@H]1Cc2ccccc2CCCCCCCCc2cncc(n2)C(=O)N1)B(O)O]

Certain embodiments of the present disclosure provide a compound of one of the following formulas and/or a pharmaceutically acceptable salt, solvate, tautomer, hydrate or prodrug derivative thereof:

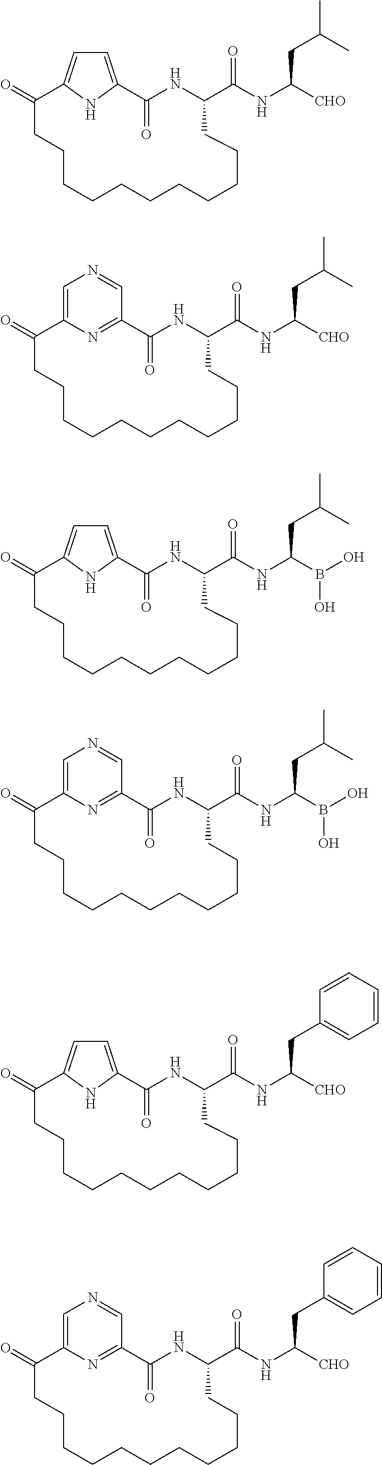 [SMILES: CC(C)C[C@@H](C=O)NC(=O)[C@@H]1CCCCCCCCCCCC(=O)c2ccc([nH]2)C(=O)N1 ; CC(C)C[C@@H](C=O)NC(=O)[C@@H]1CCCCCCCCCCCC(=O)c2cncc(n2)C(=O)N1 ; CC(C)C[C@H](NC(=O)[C@@H]1CCCCCCCCCCCC(=O)c2ccc([nH]2)C(=O)N1)B(O)O ; CC(C)C[C@H](NC(=O)[C@@H]1CCCCCCCCCCCC(=O)c2cncc(n2)C(=O)N1)B(O)O ; O=C[C@H](Cc1ccccc1)NC(=O)[C@@H]1CCCCCCCCCCCC(=O)c2ccc([nH]2)C(=O)N1 ; O=C[C@H](Cc1ccccc1)NC(=O)[C@@H]1CCCCCCCCCCCC(=O)c2cncc(n2)C(=O)N1]

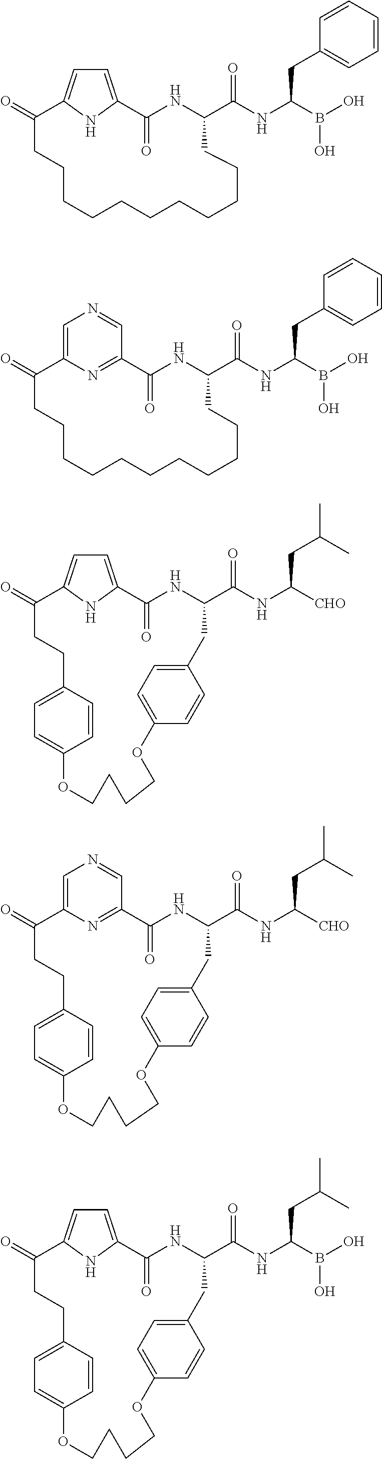 [SMILES: O=C1CCCCCCCCCCC[C@@H](C(=O)N[C@@H](Cc2ccccc2)B(O)O)NC(=O)c2ccc1[nH]2 ; O=C1CCCCCCCCCCC[C@@H](C(=O)N[C@@H](Cc2ccccc2)B(O)O)NC(=O)c2cncc1n2 ; CC(C)C[C@@H](C=O)NC(=O)[C@@H]1Cc2ccc(cc2)OCCCCOc2ccc(cc2)CCC(=O)c2ccc([nH]2)C(=O)N1 ; CC(C)C[C@@H](C=O)NC(=O)[C@@H]1Cc2ccc(cc2)OCCCCOc2ccc(cc2)CCC(=O)c2cncc(n2)C(=O)N1 ; CC(C)C[C@H](NC(=O)[C@@H]1Cc2ccc(cc2)OCCCCOc2ccc(cc2)CCC(=O)c2ccc([nH]2)C(=O)N1)B(O)O]

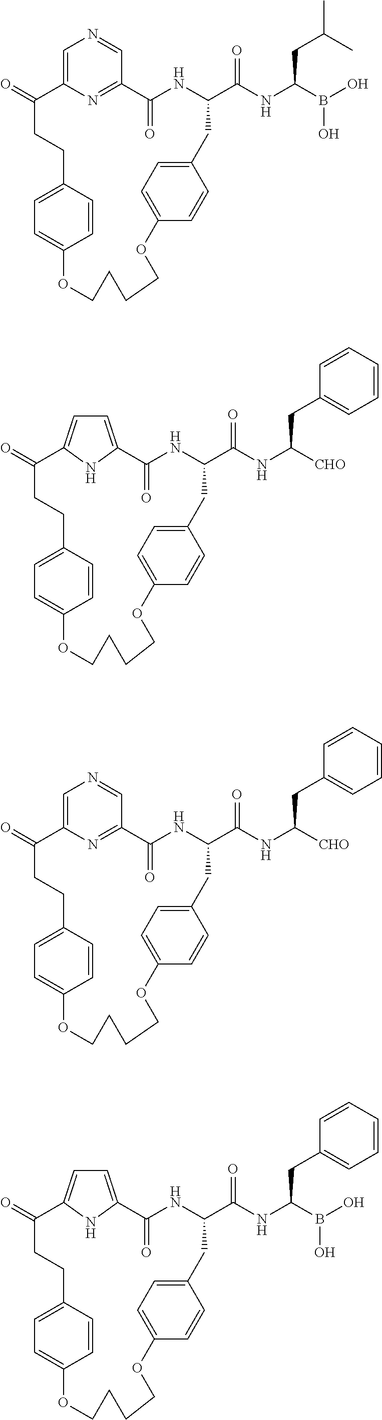 [SMILES: CC(C)C[C@H](NC(=O)[C@@H]1Cc2ccc(cc2)OCCCCOc2ccc(cc2)CCC(=O)c2cncc(n2)C(=O)N1)B(O)O ; O=C[C@H](Cc1ccccc1)NC(=O)[C@@H]1Cc2ccc(cc2)OCCCCOc2ccc(cc2)CCC(=O)c2ccc([nH]2)C(=O)N1 ; O=C[C@H](Cc1ccccc1)NC(=O)[C@@H]1Cc2ccc(cc2)OCCCCOc2ccc(cc2)CCC(=O)c2cncc(n2)C(=O)N1 ; O=C1CCc2ccc(cc2)OCCCCOc2ccc(cc2)C[C@@H](C(=O)N[C@@H](Cc2ccccc2)B(O)O)NC(=O)c2ccc1[nH]2]

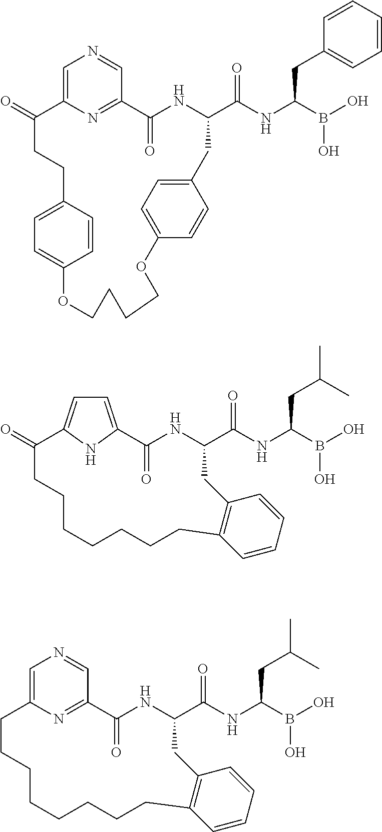 [SMILES: O=C1CCc2ccc(cc2)OCCCCOc2ccc(cc2)C[C@@H](C(=O)N[C@@H](Cc2ccccc2)B(O)O)NC(=O)c2cncc1n2 ; CC(C)C[C@H](NC(=O)[C@@H]1Cc2ccccc2CCCCCCCC(=O)c2ccc([nH]2)C(=O)N1)B(O)O ; CC(C)C[C@H](NC(=O)[C@@H]1Cc2ccccc2CCCCCCCCc2cncc(n2)C(=O)N1)B(O)O]

Certain embodiments of the present disclosure provide a compound according to Formula 1 as described with reference to any of the examples.

Certain embodiments of the present disclosure provide a macrocyclic compound as described with reference to any of the examples.

The compounds of the present disclosure may have asymmetric carbon atoms, and as such stereoisomers (both enantiomers and diastereomers) of such compounds may exist. The present disclosure contemplates pure stereoisomers and any mixture of the isomers. For example, a pure enantiomer of a compound as described herein may be isolated from a mixture of enantiomers using conventional optical resolution techniques. Enol forms and tautomers are also contemplated.

Compounds of the present disclosure may be prepared according to the general methodology described herein and in the Examples. A person skilled in the art will be able, without undue experimentation and with regard to that skill and this disclosure, to select appropriate reagents and conditions to modify these methodologies to produce compounds as described herein.

Those persons skilled in the art will also appreciate that other synthetic routes may be used to synthesize the compounds as described herein. In addition, those persons skilled in the art will appreciate that, in the course of preparing the compounds as described herein, the functional groups of intermediate compounds may need to be protected by protecting groups. Functional groups which it may be desirable to protect include, but are not limited to: hydroxyl; amino; and carboxylic acid groups. Protecting groups may be added and removed in accordance with techniques that are well known to those persons skilled in the art. The use of protecting groups is described in, for example, J. W. F. McOmie (ed.), Protective Groups in Organic Chemistry, Plenum Press, London, 1973 and T. W. Greene and P. G. M. Wutz, Protective Groups in Organic Synthesis, 2″ edition, Wiley, New York, 1991.

Methods for synthesizing compounds according to Formula I are as described herein, and in particular, with reference to the examples.

Certain embodiments of the present disclosure provide a method of synthesis of a compound according to Formula 1 as described with reference to any of the examples.

Certain embodiments of the present disclosure provide a method of synthesis of a macrocyclic compound as herein described with reference to any of the examples.

In certain embodiments, a compound as described herein has use as a protease inhibitor. In certain embodiments, the protease is a cysteine protease. In certain embodiments, the cysteine protease is a calpain. In certain embodiments, the calpain is calpain I and/or calpain II.

In certain embodiments, a compound as described herein comprises an IC50 of 250 nM or less as a protease inhibitor.

Certain embodiments of the present disclosure provide a method of inhibiting a protease, the method comprising exposing the protease to a compound as described herein. In certain embodiments, the protease is in vitro. In certain embodiments, the protease is in vivo.

The term “exposing”, and related terms such as “expose” and “exposure”, refers to directly and/or indirectly contacting and/or treating a species (for example an enzyme in vivo or in vitro, a cell in vitro or in vivo) with a compound as described herein.

For example, for a protease present in a cell in vitro, a compound as described herein may be added to a cell in culture medium, so as to expose the cell to the compound, or a derivative added to the culture medium that results in the production of the compound, thereby exposing the cell e to the compound. For a protease present in a cell in a subject, a compound as described herein may be administered to a subject to expose cells to the compound, or an derivative may be administered to a subject that results in the production of the compound in the subject, thereby exposing cells in vivo to the compound. Examples of administration routes are as described herein.

In certain embodiments, the present disclosure provide a method of inhibiting a protease in a cell, the method exposing the cell to a compound as described herein. In certain embodiments, the cell is in vitro. In certain embodiments, the cell is in vivo. In certain embodiments, the cell is an isolated cell. In certain embodiments, the cell is present in a biological system. The term “biological system refers to any cellular system. For example, the biological system may be a cell in tissue culture, a tissue or organ, or an entire animal or human subject, including a human or animal subject suffering from, or susceptible to, a disease, condition or state as described herein.

In certain embodiments, the protease or a cell is exposed to an effective amount of a compound as described herein.

The term “effective amount” as used herein refers to that amount of an agent that when exposed to a target, such as a protease or a cell, is sufficient to illicit the desired response or outcome. The effective amount will vary depending upon a number of factors, including for example the specific activity of the agent being used and the cell type.

In certain embodiments, the cell is present in vivo. In certain embodiments, the cell is present in a subject, as described herein.

Certain embodiments of the present disclosure provide a method of preventing and/or treating a disease, condition or state as described herein, the method comprising administering to the subject a therapeutically effective amount of a compound as described herein.

In certain embodiments, the disease, condition or state is a disease, condition or state associated with dysregulation of protease activity and/or dysregulation of proteosome activity.

In certain embodiments, the disease, condition or state comprises an ocular disorder, a cataract, an optic neuropathy, ischemic optic neuropathy, diabetic neuropathy, diabetic macular oedema, glaucoma, macular degeneration, retinal ischaemia, retinal damage, retinal detachment, presbyopia, an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy and a multiple myeloma,

The term “therapeutically effective amount” as used herein refers to that amount of an agent that is sufficient to effect prevention and/or treatment, when administered to a subject. The therapeutically effective amount will vary depending upon a number of factors, including for example the specific activity of the agent being used, the severity of the disease, condition or state in the subject, the age, physical condition, existence of other disease states, and nutritional status of the subject.

In certain embodiments, the compound is administered to the subject in an amount ranging from one of the following selected ranges: 1 μg/kg to 100 mg/kg; 1 μg/kg to 10 mg/kg; 1 μg/kg to 1 mg/kg; 1 μg/kg to 100 μg/kg; 1 μg/kg to 10 μg/kg; 10 μg/kg to 100 mg/kg; 10 μg/kg to 10 mg/kg; 10 μg/kg to 1 mg/kg; 10 μg/kg to 100 μg/kg; 100 μg/kg to 100 mg/kg; 100 μg/kg to 10 mg/kg; 100 μg/kg to 1 mg/kg; 1 mg/kg to 10 mg/kg; and 10 mg/kg to 100 mg/kg body weight. Other ranges are contemplated. The dose and frequency of administration may be determined by one of skill in the art.

A compound as described herein may be administered to a subject in a suitable form. In this regard, the terms “administering” or “providing” includes administering the compound, or administering a prodrug or a derivative of the compound that will form a therapeutically effective amount of the compound within the body of the subject. The terms include routes of administration that are systemic (e.g., via injection such as intravenous injection, orally in a tablet, pill, capsule, or other dosage form useful for systemic administration of pharmaceuticals), and topical (e.g., creams, solutions, and the like, including solutions such as mouthwashes, for topical oral administration).

In certain embodiments, the compound is administered orally. In certain embodiments, the compound is administered intravenously. In certain embodiments, the compound is administered via injection such as intravenous injection. In certain embodiments, the compound is administered by nebulized administration, by aerosolized administration or by being instilled into the lung.

The compound may be administered alone or may be delivered in a mixture with other therapeutic agents and/or agents that enhance, stabilise or maintain the activity of the inhibitor. In certain embodiments, an administration vehicle (e.g., pill, tablet, implant, injectable solution, etc.) would contain both the compound as described herein and additional agent(s).

When administered to a subject the therapeutically effective dosage may vary depending upon the particular compound utilized, the mode of administration, the condition, and severity thereof, as well as the various physical factors related to the subject being treated. As discussed herein, suitable daily doses range from 1 μg/kg to 100 mg/kg. The daily dosages are expected to vary with route of administration, and the nature of the compound administered. In certain embodiments the methods comprise administering to the subject escalating doses of compound and/or repeated doses. In certain embodiments, the compound is administered orally. In certain embodiments, the compound is administered via injection, such as intravenous injection. In certain embodiments, the compound is administered parenterally. In certain embodiments, the compound is administered by direct introduction to the lungs, such as by aerosol administration, by nebulized administration, and by being instilled into the lung. In certain embodiments, the compound is administered by implant. In certain embodiments, the compound is administered by subcutaneous injection, intraarticularly, rectally, intranasally, intraocularly, vaginally, or transdermally.

“Intravenous administration” is the administration of substances directly into a vein.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

In certain embodiments, the compound is administered as an immediate release formulation. The term “immediate release formulation” is a formulation which is designed to quickly release a therapeutic agent in the body over a shortened period of time.

In certain embodiments, the compound is administered as a sustained release formulation. The term “sustained release formulation” is a formulation which is designed to slowly release a therapeutic agent in the body over an extended period of time.

In certain embodiments, a compound as described herein may be used in a pharmaceutical composition. In certain embodiments, a compound as described herein may be used in a pharmaceutical composition for use in the methods of the present disclosure as described herein.

Certain embodiments of the present disclosure provide a pharmaceutical composition comprising a therapeutically effective amount of a compound as described herein.

Certain embodiments of the present disclosure provide use of a compound as described herein in the preparation of a medicament.

In certain embodiments, the pharmaceutical composition further comprises a pharmaceutically acceptable carrier.

Certain embodiments of the present disclosure provide use of a compound as described herein in the preparation of a medicament.

Certain embodiments of the present disclosure provide use of a compound as described herein in the preparation of a medicament for preventing and/or treating a disease, condition or state as described herein.

In certain embodiments, the medicament is suitable for delivery to the subject by one or more of intravenous administration, intratracheal administration, by nebulized administration, by aerosolized administration, by instillation into the lung, by oral administration, by parenteral administration, by implant, by subcutaneous injection, intraarticularly, rectally, intranasally, intraocularly, vaginally, or transdermally.

In certain embodiments, a compound as described herein is provided in a pharmaceutically acceptable carrier suitable for administering the pharmaceutical composition to a subject. The carriers may be chosen based on the route of administration as described herein, the location of the target issue, the inhibitor being delivered, the time course of delivery of the drug, etc. The term “pharmaceutically acceptable carrier” refers to a substantially inert solid, semi-solid or liquid filler, diluent, encapsulating material or formulation auxiliary of any type. An example of a pharmaceutically acceptable carrier is physiological saline. Other physiologically acceptable carriers and their formulations are known in the art. Some examples of materials which can serve as pharmaceutically acceptable carriers include, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN 80; buffering agents such as magnesium hydroxide and aluminium hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present.

In certain embodiments, a compound as described herein may be administered or present in a pharmaceutical composition as a pharmaceutically acceptable salt. The term “pharmaceutically acceptable salt” refers to acid addition salts or metal complexes which are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like.

In certain embodiments, the pharmaceutical compositions or medicament comprises other therapeutic agents and/or agents that enhance, stabilise or maintain the activity of the active.

Oral formulations containing the compounds as described herein may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminium silicate, and triethanolamine. Oral formulations may utilize standard delay or time-release formulations to alter the absorption of the peptides. The oral formulation may also consist of administering the active ingredient in water or a fruit juice, containing appropriate solubilizers or emulsifiers as needed.

In certain embodiments, it may be desirable to administer a compound as described herein directly to the airways in the form of an aerosol. Formulations for the administration of aerosol forms are known.

In certain embodiments, a compound as described herein may also be administered parenterally (such as directly into the joint space) or intraperitoneally. For example, solutions or suspensions of these compounds in a non-ionised form or as a pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations typically contain a preservative to prevent the growth of microorganisms.

In certain embodiments, a compound as described herein may also be administered by injection. Pharmaceutical forms suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol and liquid polyethylene glycol), suitable mixtures thereof, and vegetable oils.

In certain embodiments, a compound as described herein may also be administered intravenously. Compositions containing the inhibitor described herein suitable for intravenous administration may be formulated by a skilled person.

In certain embodiments, a compound as described herein may also be administered transdermally. Transdermal administrations are understood to include all administrations across the surface of the body and the inner linings of bodily passages including epithelial and mucosal tissues. Such administrations may be carried out using the inhibitor as described herein, or pharmaceutically acceptable salts thereof, in lotions, creams, foams, patches, suspensions, solutions, and suppositories (rectal and vaginal).

Transdermal administration may also be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient.

In certain embodiments, a compound as described herein may also be administered by way of a suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

Additional numerous various excipients, dosage forms, dispersing agents and the like that are suitable for use in connection with the administration of a compound as described herein and/or the formulation into medicaments or pharmaceutical compositions. Formulations are known and described in, for example, Remington's Pharmaceutical Sciences, 17th ed., Mack Publishing Company, Easton, Pa., 1985, which is incorporated herein by reference in its entirety.

Certain embodiments of the present disclosure provide a method of preventing and/or treating a disease, condition or state in a subject associated with dysregulation of protease activity and/or dysregulation of proteosome activity, the method comprising administering to the subject a therapeutically effective amount of one or more compounds as described herein.

In certain embodiments, the disease, condition or state is associated with dysregulation of cysteine protease activity.

In certain embodiments, the disease, condition or state is associated with dysregulation of activity of a calpain.

In certain embodiments, the disease, condition or state comprises one or more of an ocular disorder, a cataract, an optic neuropathy, ischemic optic neuropathy, diabetic neuropathy, diabetic macular oedema, glaucoma, macular degeneration, retinal ischaemia, retinal damage, retinal detachment, and presbyopia.

In certain embodiments, the disease, condition or state comprises one or more of an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy, and a multiple myeloma.

Certain embodiments of the present disclosure provide a method of preventing and/or treating an ocular disorder, a cataract, an optic neuropathy, ischemic optic neuropathy, diabetic neuropathy, diabetic macular oedema, glaucoma, macular degeneration, retinal ischaemia, retinal damage, retinal detachment, presbyopia, an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy and a multiple myeloma, the method comprising administering to the subject a therapeutically effective dose of a compound of Formula I and/or a pharmaceutically acceptable salt, solvate, tautomer, hydrate or prodrug derivative thereof:

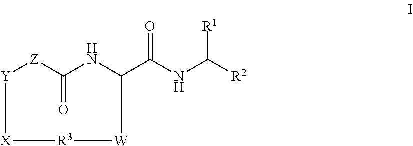

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

Compounds according to Formula I as are described herein,

Certain embodiments of the present disclosure provide a method of identifying a protease inhibitor.

Certain embodiments of the present disclosure provide a method of identifying a protease inhibitor, the method comprising:
(i) providing a P1-P3 or a P2-P4 macrocyclic peptidomimetic compound;
(ii) determining the ability of the P1-P3 or the P2-P4 macrocyclic peptidomimetic compound to inhibit a protease; and
(iii) identifying the P1-P3 or the P2-P4 macrocyclic peptidomimetic compound as a protease inhibitor.

In certain embodiments, the protease comprises a cysteine protease.

In certain embodiments, the cysteine protease comprises a calpain.

In certain embodiments, the calpain comprises calpain I and/or calpain II.

In certain embodiments, the P1-P3 macrocyclic peptidomimetic compound comprises a compound according to Formula I, as described herein.

In certain embodiments, the determining of the ability of the P1-P3 or the P2-P4 macrocyclic peptidomimetic compound to inhibit a protease comprises determining the ability of the P1-P3 or the P2-P4 macrocyclic peptidomimetic compound to inhibit a protease in vitro.

In certain embodiments, the determining of the ability of the P1-P3 or the P2-P4 macrocyclic peptidomimetic compound to inhibit a protease comprises determining the ability of the P1-P3 or the P2-P4 macrocyclic peptidomimetic compound to inhibit a protease in vivo.

Methods for identifying a protease inhibitor are as described herein, and in particular, with reference to the examples.

Certain embodiments of the present disclosure provide a method of identifying a therapeutic agent for preventing and/or treating a disease, condition or state associated with dysregulation of protease activity and/or dysregulation of proteosome activity.

Certain embodiments of the present disclosure provide a method of identifying a therapeutic agent for preventing and/or treating a disease, condition or state associated with dysregulation of protease activity and/or dysregulation of proteosome activity, the method comprising:
(i) providing a P1-P3 or a P2-P4 macrocyclic peptidomimetic compound;
(ii) determining the ability of the P1-P3 or the P2-P4 macrocyclic peptidomimetic compound to prevent and/or treat a disease, condition or state associated with dysregulation of protease activity and/or dysregulation of proteosome activity; and
(iii) identifying the P1-P3 or the P2-P4 macrocyclic peptidomimetic compound as a therapeutic agent for preventing and/or treating a disease, condition or state associated with dysregulation of protease activity and/or dysregulation of proteosome activity.

Diseases, conditions or states associated with dysregulation of protease activity and/or dysregulation of proteosome activity are as described herein.

In certain embodiments, the disease, condition or state is associated with dysregulation of activity of a calpain.

In certain embodiments, the disease, condition or state is associated with dysregulation of activity of calpain I and/or calpain II

In certain embodiments, the disease, condition or state comprises an ocular disorder, a cataract, an optic neuropathy, ischemic optic neuropathy, diabetic neuropathy, diabetic macular oedema, glaucoma, macular degeneration, retinal ischaemia, retinal damage, retinal detachment, or presbyopia.

In certain embodiments the P1-P3 macrocyclic peptidomimetic compound comprises a compound with Formula I and/or a pharmaceutically acceptable salt, solvate, tautomer, hydrate or prodrug derivative thereof:

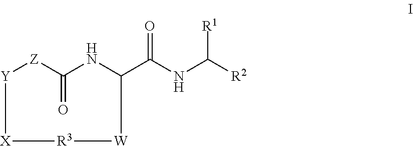

wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

Compounds according to Formula I are a described herein.

Methods for determining the ability of the P1-P3 or the P2-P4 macrocyclic peptidomimetic compound to prevent and/or treat a disease, condition or state associated with dysregulation of protease activity and/or dysregulation of proteosome activity include the use of animal models.

Certain embodiments of the present disclosure provide a method of identifying a protease inhibitor as described herein with reference to any of the examples.

Certain exemplary embodiments are illustrated by some of the following examples. It is to be understood that the following description is for the purpose of describing particular embodiments only and is not intended to be limiting with respect to the above description.

### Example 1

### Calpain Assays

Fluorometric assays (excitation: 485 nm, emission: 520 nm) with ovine calpains 1 and 2 were done with a (BMG Labtech) Fluostar Optima plate reader at 37.0±0.2° C. in 96-well black (Greiner Bio-one) microassay plates. Calpains 1 and 2, partially purified from sheep lung by hydrophobic interaction and ion-exchange chromatography, were diluted in 20 mM MOPS, pH 7.5, containing 2 mM EGTA, 2 mM EDTA and 0.035% v/v 2-mercaptoethanol to give a linear response over the course of the assay. The substrate BODIPY-F1 casein was prepared as reported. {1} A 0.0005% solution of the substrate in 10 mM MOPS, pH 7.5, 10 mM CaCl₂, 0.1 mM NaN₃, 0.1% v/v 2-mercaptoethanol was prepared freshly before each experiment. Stock solutions of inhibitors (5 mM) were freshly prepared in DMSO and diluted in DMSO/water mixtures to obtain a total DMSO concentration of 4% v/v.

Inhibition studies were performed in the presence of seven different inhibitor concentrations and 1% v/v DMSO in a volume of 200 μL: 50 μl of inhibitor solution was added to a microassay well followed by 50 μl of calpain-containing solution. The reaction was initiated by adding 100 μl of BODIPY-F1 casein solution to each well and progress curves were monitored every 30 s over 570 s. Uninhibited enzyme activity was determined by adding 4% v/v DMSO in water instead of inhibitor solution. Every experiment included two blanks, a Ca²⁺ blank and an EDTA blank. The Ca²⁺ blank contained 50 μl water and 50 μl 20 mM MOPS, pH 7.5, 2 mM EGTA, 2 mM EDTA and 0.035% v/v 2-mercaptoethanol instead of inhibitor and enzyme solution, respectively. For the EDTA blank, 50 μl 50 mM EDTA/NaOH, pH 7.5, was added instead of inhibitor solution to the well.

The rate of enzyme-catalyzed substrate hydrolysis was obtained by linear regression of the progress curves over the time course. If slow-binding inhibition occurred {2}, only those data points representing the steady state of enzyme-inhibitor interaction were taken into account, i.e. data points between 390 s and 570 s. The rate of the enzymatic reaction was corrected by the average value of the rates obtained for the two blanks, and the rate in the absence of inhibitor was set to 100%.

The average value of rates obtained in two separate experiments, each in triplicate, was plotted versus the inhibitor concentration and IC50 values were calculated with the following equation:

$${v}_{i} = \frac{{v}_{0}}{1 + \frac{[ I ]}{{IC}_{50}}}$$

where [I] is the inhibitor concentration, and ν₀ and ν are the enzyme activities in the absence and presence of inhibitor. All analyses were done with the program GraphPad Prism version 5.02 for Windows, GraphPad Software, San Diego Calif. USA, www.graphpad.com.

### Example 2

### Bovine Chymotrypsin Assays

The activity of bCT was assayed spectrophotometrically with a Varian Cary 5000 UV-VIS-NIR spectrophotometer equipped with a thermostated multicell holder at 25.0±0.1° C. Assay buffer was: Tris-HCl (77 mM), CaCl₂ (20 mM), pH 7.8 (pH optimum of α-chymotrypsin {3}). A solution of bCT (21.9 μg/mL) in aq HCl (1 mM) was prepared daily by a 1:40 dilution of a stock solution (874 μg/mL) in aq HCl (1 mM) and kept at 0° C. A 1:100 dilution in ice-cold aq HCl (1 mM) was prepared immediately before starting each measurement. Stock solutions of the substrate Suc-Ala-Ala-Pro-Phe-pNA (10 mM) and all inhibitors (5 mM) were freshly prepared in DMSO and stored at r.t. All compounds were analyzed at five different inhibitor concentrations, [I]. Progress curves were monitored at 405 nm over 6 min and characterized by a linear steady state turnover of the substrate.

The Inhibition studies were performed in the presence of 6% v/v DMSO in a volume of 1 mL containing 0.011 μg/mL bCT, different concentrations of Suc-Ala-Ala-Pro-Phe-pNA (10-100 μM) and inhibitor. Into a cuvette containing 890 μL assay buffer, 10 μL of substrate solution (1-10 mM), inhibitor stock, and DMSO were added to give a total volume of 950 mL. After thoroughly mixing the contents of the cuvette, the enzymatic reaction was initiated by adding 50 μL of bCT solution. Uninhibited enzyme activity was determined by adding DMSO instead of inhibitor solution. Non-enzymatic hydrolysis of Suc-Ala-Ala-Pro-Phe-pNA was analyzed by adding DMSO and 1 mM aqueous HCl instead of inhibitor and enzyme solution, respectively, and found to be negligible. The rate of enzyme-catalyzed hydrolysis of 100 μM substrate was determined without inhibitor in each experiment and was set to 100%. Kᵢ values of inhibitors were determined graphically according to Dixon {4} using mean values of percentage rates obtained in three separate experiments at two different substrate concentrations, [S].

General Methods:

¹H NMR (600 MHz) and ¹³C NMR (150 MHz) spectra were recorded on a Varian Inova 600 MHz spectrometer. All NMR spectra were measured in CDCl₃ or DMSO-d₆. Chemical shifts are reported in parts per million (ppm, 8). Chemical shifts of CDCl₃ (δ_{C}=77.00 ppm), DMSO-d₆ (δ_{C}=39.70 ppm), or tetramethylsilane (TMS, δ_{H}=0.00 ppm) were used as internal standards in ¹³C NMR experiments and ¹H NMR experiments. Molecular masses were determined by electrospray ionization (ESI) mass spectrometry on a Finnigan LCQ Ion Trap mass spectrometer, conditions were as follows: needle potential, 4500 V; tube lens, 60 V; heated capillary, 200° C., 30 V; sheath gas flow, 30 psi. Optical rotation measurements were performed on a ATAGO AP-100 polarimeter with 9.99 mm path length. Measurements were taken at 23° C. in DMSO at λ=589 nm. [α]_{D} values are given in units of mL/g·dm and the sample concentration given in units of 10 mg/mL. Melting points were obtained on a Reichert Thermovar Kofler apparatus, and are uncorrected. Thin-layer chromatography was carried out on Merck aluminium sheets with silica gel 60 F₂₅₄, and visualized with a Vilber Lourmat VL-6C (6W-254 nm tube). Flash column chromatography was performed using Scharlau silica gel 60 (230-400 mesh). All yields reported are isolated yields, determined to be pure by NMR spectroscopy. Amino acids had (L)-configuration unless otherwise stated. Boc-Allyl-Gly was purchased from Boaopharma Inc. (L)-Phenalaninol, HATU and EDCI were purchased from GL Biochem (Shanghai) Limited. Boc-Try(All)-OH, (L)-Leucinol and Dess Martin Periodinane was purchased from Chem-Impex International, Inc. (CII). All other reagents and chemicals were purchased from Sigma-Alrdich.

Friedel-Craft's Acylation:

To the respective acid chloride (2 equiv.) in nitromethane (6 mL/1 g of acid chloride) was added the respective pyrrole (1 equiv.) followed by the addition of ytterbium (III) trifluoromethanesulfonate (0.1 equiv.). The resulting dark red solution was stirred at ambient temperature for 21 h. The reaction was quenched by addition of saturated aqueous NaHCO₃ and the mixture was extracted with diethyl ether (3×). The organic layers were combined, washed with saturated aqueous NaHCO₃, H₂O (2×), brine, dried over MgSO₄ and volatiles were removed in vacuo and the resultant crude oil was purified via flash chromatography to give the desired pure pyrroles.

General Procedure B:

Ester hydrolysis with KOH: To a solution of respective ester (1.0 equiv.) in 1:1 THF/H₂O (20 mL/1 g ester) was added potassium hydroxide (8.0 equiv) in one portion and stirred at 40-50° C. for 18 h. The reaction mixture was cooled and partitioned between diethyl ether and water. The aqueous layer was collected, cooled to 0° C. and acidified to pH 1 with 32% aqueous HCl. The precipitate was collected, washed with water and volatiles removed in vacuo at ambient temperature overnight to give the desired carboxylic acids.

General Procedure C: HATU Mediated Peptide Coupling:

To the respective carboxylic acid (1.0 equiv.) in dry dichloromethane (45 mL/1 g of carboxylic acid) was added the appropriate amine (1.15 equiv.), HATU (1.2 equiv) and HOBt (1.2 equiv). The reaction mixture was stirred for 5 min at ambient temperature under a nitrogen atmosphere. To the solution wad added DIPEA (4.0 equiv) and stirred under a nitrogen atmosphere for 18 h. The reaction mixture was partitioned between dichloromethane and 1M aqueous HCl. The organic phase was separated and washed with saturated NaHCO₃, water (2×) and brine. The organic phase was dried over Na₂SO₄, filtered and concentrated in vacuo. The crude product was purified by flash chromatography to yield the desired pure amides.

General Procedure D: EDCI Mediated Peptide Coupling:

To the respective acid (1.0 equiv.) in dry dichloromethane (50 mL/1 g of carboxylic acid) was added the appropriate amine (1.15 equiv.), EDCI (1.4 equiv) and HOBt (1.5 equiv). The reaction mixture was stirred for 5 min at ambient temperature under a nitrogen atmosphere. To the solution was added DIPEA (2.6 equiv) and stirred under a nitrogen atmosphere for 18 h. The reaction mixture was partitioned between dichloromethane and 2M aqueous HCl. The organic phase was separated and washed with 2M aqueous HCl (2×), water (2×) and brine (10 mL). The organic phase was dried over Na₂SO₄, filtered and concentrated in vacuo. The crude product was purified by flash chromatography to yield the desired pure amides.

General Procedure E: Ring Closing Metathesis (Thermal Reflux):

To the respective acyclic diene (1.0 equiv.) in anhydrous dichloromethane (2.5 mL/1 mg of acyclic diene) was added Grubb's 2^{nd} generation catalyst (10 mol %). The mixture was heated to 45° C. and stirred for 30 min under a nitrogen atmosphere. An additional portion of Grubb's 2^{nd} generation catalyst (10 mol %) was added and the solution was stirred for 18 h at 45° C. The reaction was quenched by addition of activated charcoal and stirred for 18 h at ambient temperature. Volatiles were removed in vacuo and the crude residue was purified by flash chromatography to give the desired pure macrocycles.

General Procedure F: Hydrogenation of a Carbon-Carbon Double Bond:

To the respective olefin (1.0 equiv.) in ethyl acetate (400 mL/1 g of olefin) was added palladium on carbon catalyst (10%, 2.0 equiv.). The mixture was stirred under a hydrogen atmosphere at ambient temperature and atmospheric pressure for 18 h. The suspension was filtered through Celite, concentrated in vacuo and the crude residue was purified by flash chromatography to give the desired alkanes.

General Procedure G: Ester Hydrolysis with NaOH:

To a solution of respective ester (1.0 equiv.) in 1:1 THF/H₂O (20 mL/1 g ester) was added 2M sodium hydroxide (8.0 equiv) in one portion and stirred at room temperature for 18 h. THF was removed in vacuo. The resulting solution was neutralized with 32% aqueous HCl and lyophilized to give the desired carboxylic acids, which was used in the next step without purification.

General Procedure H: Oxidation of an Amino Alcohol:

To a solution of respective amino alcohol (1.0 equiv.) in anhydrous DCM (70 mL/1 g amino alcohol), was added Dess Martin Periodinane (2.0 equiv) and stirred for 1 h at room temperature under a nitrogen atmosphere. The reaction was quenched by addition of saturated aqueous NaHCO₃ and Na₂S₂O₅ and stirred at room temperature for 15 min. The reaction mixture was extracted with DCM (2×), dried over Na₂SO₄ and volatiles were removed in vacuo. The resultant crude was purified by HPLC to give the desired aldehydes.

### (S)—N—((S)-1-hydroxy-4-methylpentan-2-yl)-2,16-dioxo-3,20-diazabicyclo[15.2.1]icosa-1(19),17-diene-4-carboxamide 1a

Macrocyclic ester 16 (61 mg, 0.17 mmol) was hydrolized (General Procedure G) to the carboxylic acid, which was coupled to (L)-Leucinol (General Procedure D) and the crude product was purified by flash chromatography on silica using a gradient of ethtyl acetate and (50/70) petroleum ether to give 1a (42 mg, 62%) as a white oil. ¹H NMR (CDCl₃, 600 MHz) δ 0.84 (d, J=6.0 Hz, 3H, CH₂CH(CH₃)₂), 0.88 (d, J=7.2 Hz, 3H, CH₂CH(CH₃)₂), 0.85-1.35 (m, 18H, CO(CH₂)₂(CH₂)₈), CH₂CH(CH₃)₂), 1.58-1.59 (m, 2H, CO(CH₂)₁₀CHH, CH₂CH(CH₃)₂), 1.66-1.67 (m, 2H, COCH₂CH₂), 1.77-1.79 (m, 1H, CO(CH₂)₁₀CHH), 2.61-2.62 (m, 1H, COCHH), 2.88-2.90 (m, 1H, COCHH), 3.20-3.24 (m, 1H, CHHOH), 3.28-3.33 (m, 1H, CHHOH), 3.81 (br d, J=4.2 Hz, 1H, NHCHCH₂OH), 4.48 (t, J=8.7 Hz, 1H, NHCHCO), 4.62 (t, J=5.1 Hz, 1H, CH₂OH), 6.81 (s, 1H, pyrrole H), 6.91 (s, 1H, pyrrole H), 7.66 (d, J=8.4 Hz, 1H, NHCHCH₂OH), 8.41 (d, J=8.4 Hz, 1H, NHCHCO), 12.16 (br s, 1H, pyrrole NH); ¹³C NMR (CDCl₃, 150 MHz) δ 21.9, 23.4, 24.2, 25.9, 26.9, 27.3, 27.5, 27.7, 27.9, 28.1, 28.6, 31.5, 48.7, 52.6, 63.8, 114.0, 115.0, 131.0, 134.0, 159.1, 171.5, 193.0; HRMS (ES) 448.3149 (MH⁺); C₂₅H₄₂N₃O₄ requires 448.3170; [α]_{D}=+1.3 (c 0.2 in (CH₃)₂SO).

### (S)—N—((S)-1-hydroxy-3-phenylpropan-2-yl)-2,16-dioxo-3,20-diazabicyclo[15.2.1]icosa-1(19),17-diene-4-carboxamide 1b

Macrocyclic ester 16 (79 mg, 0.22 mmol) was hydrolized (General Procedure G) to the carboxylic acid, which was coupled to (L)-Phenalaninol (General Procedure D) and the crude product was purified by flash chromatography on silica using a gradient of ethtyl acetate and (50/70) petroleum ether to give 1b (62 mg, 58%) as a white oil. ¹H NMR (CDCl₃, 600 MHz) δ 0.64-1.47 (m, 16H, CO(CH₂)₂(CH₂)₈), 1.74-1.80 (m, 1H, CO(CH₂)₁₀CHH), 1.81-1.86 (m, 2H, COCH₂CH₂), 2.12 (br s, 1H, CO(CH₂)₁₀CHH), 2.51 (br s, 1H, COCHH), 2.88-2.92 (m, 2H, CH₂Ar), 3.08 (br s, 1H, COCHH), 3.77 (s, 2H, CH₂OH), 4.10-4.18 (m, 1H, CH₂OH), 4.24 (q, J=8.0 Hz, 1H, NHCHCH₂OH), 5.36 (br s, 1H, NHCHCO), 6.74 (s, 1H, pyrrole H), 6.95-6.98 (m, 2H, pyrrole H, NHCH), 7.14-7.26 (m, 5H, ArH), 8.19 (br s, 1H, NHCH), 11.42 (br s, 1H, pyrrole NH); ¹³C NMR (CDCl₃, 150 MHz) δ 24.9, 26.9, 27.3, 28.3, 29.0, 29.2, 29.5, 29.6, 29.8, 37.4, 39.1, 52.5, 53.6, 62.5, 118.0, 126.5, 128.6, 129.5, 131.1, 134.1, 138.4, 159.8, 171.9, 195.6; HRMS (ES) 482.2991 (MH⁺); C₂₈H₄₀N₃O₄ requires 482.3013; [α]_{D}=+1.6 (c 0.2 in (CH₃)₂SO).\

### (S)—N—((S)-4-methyl-1-oxopentan-2-yl)-2,16-dioxo-3,20-diazabicyclo[15.2.1]icosa-1(19),17-diene-4-carboxamide 1c

Alcohol 1a (35 mg, 0.078 mmol) was oxidized (General Procedure H) and the crude product was purified by rp-HPLC to give 1c (13 mg, 37%) as a clear oil. ¹H NMR (CDCl₃, 600 MHz) δ 0.79-1.35 (m, 17H, CO(CH₂)₂CHH(CH₂)₇), 0.94 (t, J=5.4 Hz, 6H, CH₂CH(CH₃)₂), 1.38-1.44 (m, 1H, CO(CH₂)₂CHH), 1.46-1.50 (m, 1H, CHHCH(CH₃)₂), 1.69-1.76 (m, 2H, CHHCH(CH₃)₂), 1.66-1.67 (m, 2H, COCH₂CH₂(CH₂)₈CHH), 2.07-2.10 (m, 1H, CO(CH₂)₁₀CHH), 2.69 (br s, 1H, COCHH(CH₂)₁₀), 2.88 (br s, 1H, COCHH(CH₂)₁₀, 4.52-4.55 (m, 1H, NHCHCHO), 4.89-4.90 (m, 1H, NHCHCO), 6.80 (s, 1H, pyrrole H), 6.93 (s, 1H, pyrrole H), 7.09 (br s, 2H, 2×NH), 9.59 (s, 1H, CHO), 10.87 (br s, 1H, pyrrole NH); ¹³C NMR (CDCl₃, 150 MHz) δ 22.0, 23.2, 24.7, 25.0, 26.8, 28.5, 29.0, 29.3, 29.5, 31.0, 37.8, 39.3, 53.5, 57.6, 112.0, 116.8, 130.5, 134.5, 160.3, 172.6, 194.6, 199.2; HRMS (ES) 446.3001 (MH⁺); C₂₈H₄₀N₃O₄ requires 446.3013; [α]_{D}=+2.2 (c 0.2 in (CH₃)₂SO).

### (S)-2,16-dioxo-N—((S)-1-oxo-3-phenylpropan-2-yl)-3,20-diazabicyclo[15.2.1]icosa-1(19),17-diene-4-carboxamide 1d

Alcohol 1b (58 mg, 0.12 mmol) was oxidized (General Procedure H) and the crude product was purified by rp-HPLC to give 1d (20 mg, 34%) as a clear oil. ¹H NMR (CDCl₃, 600 MHz) δ 0.78-1.29 (m, 15H, CO(CH₂)₂(CH₂)₇CHH), 1.38-1.40 (m, 1H, CO(CH₂)₉CHH), 1.68-1.86 (m, 3H, COCH₂CH₂(CH₂)₈CHH), 2.00-2.04 (m, 1H, CO(CH₂)₁₀CHH), 2.69 (br s, 1H, COCHH), 2.85 (br s, 1H, COCHH), 3.08-3.12 (m, 1H, CHHAr), 3.17-3.20 (CHHAr), 4.73 (q, J=7.0 Hz, 1H, NHCHCHO), 4.81 (br s, 1H, NHCHCO), 6.77 (s, 1H, pyrrole H), 6.91 (br s, 2H, pyrrole H, NHCH), 7.06-7.22 (m, 6H, ArH, NHCH), 9.66 (s, 1H, CHO), 10.82 (br s, 1H, pyrrole NH); ¹³C NMR (CDCl₃, 150 MHz) δ 24.7, 26.8, 28.2, 28.5, 29.0, 29.3, 29.5, 29.9, 30.5, 31.1, 35.2, 39.3, 53.3, 60.0, 116.9, 127.3, 128.9, 129.4, 130.4, 134.5, 135.6, 160.2, 172.3, 194.5, 198.7; HRMS (ES) 480.2834 (MH⁺); C₂₈H₃₈N₃O₄ requires 480.2857; [α]_{D}=+1.8 (c 0.2 in (CH₃)₂SO).

XXX 2a

Macrocyclic ester 17 (29 mg, 0.075 mmol) was hydrolized (General Procedure G) to the carboxylic acid, which was coupled to (L)-Leucinol (General Procedure D) and the crude product was purified by flash chromatography on silica using a gradient of ethtyl acetate and (50/70) petroleum ether to give 2a (30 mg, 83%) as a white oil. ¹H NMR (CDCl₃, 600 MHz) δ 0.87 (d, J=2.4 Hz, 3H, CH₂CH(CH₃)₂), 0.89 (d, J=2.4 Hz, 3H, CH₂CH(CH₃)₂), 1.33-1.43 (m, 2H, CH₂CH(CH₃)₂), 1.44-1.66 (m, 4H, CH₂CH(CH₃)₂, OCH₂CHHCH₂CH₂), 1.70-1.76 (m, 1H, OCH₂CHH(CH₂)₂), 1.80-1.86 (m, 1H, O(CH₂)₃CHH), 2.20-2.26 (m, 1H, O(CH₂)₃CHH), 2.89-2.95 (m, 4H, ArCHHCH₂, CH₂OH), 3.00-3.04 (m, 1H, ArCHHCH₂), 3.66-3.67 (m, 1H, CHHOH), 3.75-3.77 (m, 1H, CHHOH), 3.89-3.93 (m, 1H, OCHH(CH₂)₃), 3.96-4.00 (m, 1H, OCHH(CH₂)₃), 4.07-4.09 (m, 1H, NHCHCH₂OH), 4.79-4.80 (m, 1H, NHCHCO), 6.38-6.39 (m, 1H, pyrrole H), 6.52 (br s, 2H, OArH), 6.62 (s, 1H, pyrrole H), 6.72 (d, J=7.8 Hz, 1H, NHCHCO), 6.78 (d, J=7.2 Hz, 1H, NHCHCH₂OH), 6.85 (br s, 2H, OArH), 9.13 (br s, 1H, pyrrole NH); ¹³C NMR (CDCl₃, 150 MHz) δ 22.6, 23.0, 25.1, 29.0, 29.2, 29.5, 33.9, 38.7, 40.4, 49.1, 52.6, 64.8, 110.3, 114.3, 117.2, 118.7, 130.7, 133.2, 133.4, 139.3, 159.7, 171.7, 193.0; HRMS (ES) 470.2636 (MH⁺); C₂₆H₃₆N₃O₅ requires 470.2649; [α]_{D}=+1.4 (c 0.1 in (CH₃)₂SO).

XXX 2b

Macrocyclic ester 17 (69 mg, 0.18 mmol) was hydrolized (General Procedure G) to the carboxylic acid, which was coupled to (L)-Phenalaninol (General Procedure D) and the crude product was purified by flash chromatography on silica using a gradient of ethtyl acetate and (50/70) petroleum ether to give 2b (32 mg, 36%) as a white oil. ¹H NMR (CDCl₃, 600 MHz) δ 1.42-1.55 (m, 2H, O(CH₂)₂CH₂CH₂), 1.59-1.62 (m, 1H, OCH₂CHH(CH₂)₂), 1.72-1.82 (m, 2H, OCH₂CHHCH₂CHH), 2.19-2.22 (m, 1H, OCH₂CH₂CH₂CHH), 2.83-2.91 (m, 3H, ArCHHCH₂, CHCH₂Ar), 2.92-3.03 (m, 3H, ArCHHCH₂), 3.17 (br s, 1H, CH₂OH), 3.69-3.75 (m, 2H, CH₂OH), 3.87-3.90 (m, 1H, OCHH(CH₂)₃), 3.94-3.97 (m, 1H, OCHH(CH₂)₃), 4.20-4.21 (m, 1H, CHCH₂Ar), 4.88 (br s, 1H, NHCHCO), 6.33-6.34 (m, 1H, pyrrole H), 6.53 (br s, 2H, OArH), 6.58-6.60 (m, 2H, NHCHCO, pyrrole H), 6.82 (br s, 2H, OArH), 7.16-7.26 (m, 6H, 5×ArH, NHCHCH₂OH), 9.43 (br s, 1H, pyrrole NH); ¹³C NMR (CDCl₃, 150 MHz) δ 20.1, 28.2, 31.2, 34.0, 37.2, 41.9, 52.8, 63.3, 66.0, 110.0, 117.2, 126.8, 128.7, 128.8, 129.3, 129.4, 131.2, 135.5, 137.8, 157.4, 159.6, 171.3, 193.4; HRMS (ES) 504.2481 (MH⁺); C₂₉H₃₄N₃O₅ requires 504.2493; [α]_{D}=+1.3 (c 0.2 in (CH₃)₂SO).

XXX 2c

Alcohol 2a (15 mg, 0.032 mmol) was oxidized (General Procedure H) and the crude product was purified by rp-HPLC to give 2c (6 mg, 37%) as a clear oil. ¹H NMR (CDCl₃, 600 MHz) δ 0.96-1.00 (m, 6H, CH₂CH(CH₃)₂), 1.41-1.55 (m, 3H, O(CH₂)₂CH₂CH₂, CHHCH(CH₃)₂), 1.69-1.77 (m, 4H, CHHCH(CH₃)₂, OCH₂CH₂(CH₂)₂), 1.77-1.84 (m, 1H, O(CH₂)₃CHH), 2.28-2.32 (m, 1H, O(CH₂)₃CHH), 2.86-2.95 (m, 3H, ArCHHCH₂), 3.04-3.06 (m, 1H, ArCHHCH₂), 3.90-3.95 (m, 1H, OCHH(CH₂)₃), 4.02-4.07 (m, 1H, OCHH(CH₂)₃), 4.59-4.63 (m, 1H, NHCHCHO), 4.65-4.68 (m, 1H, NHCHCO), 6.21 (d, J=6.6 Hz, 1H, NHCHCHO), 6.36-6.37 (m, 1H, pyrrole H), 6.41 (br s, 2H, OArH), 6.52 (d, J=7.2 Hz, 1H, NHCHCO), 6.70 (br s, 2H, OArH), 6.71-6.72 (m, 1H, pyrrole H), 8.41 (br s, 1H, pyrrole NH), 9.60 (s, 1H, CHO); ¹³C NMR (CDCl₃, 150 MHz) δ 19.6, 22.1, 23.2, 25.1, 27.6, 30.6, 34.0, 38.0, 42.3, 57.6, 65.7, 109.8, 115.9, 129.1, 131.7, 135.2, 157.4, 171.4, 192.1, 198.8; HRMS (ES) 468.2481 (MH⁺); C₂₆H₃₄N₃O₅ requires 468.2493; [α]_{D}=+1.8 (c 0.1 in (CH₃)₂SO).

XXX 2d

Alcohol 2b (26 mg, 0.05 mmol) was oxidized (General Procedure H) and the crude product was purified by rp-HPLC to give 2d (4 mg, 15%) as a clear oil. ¹H NMR (CDCl₃, 600 MHz) δ 1.39-1.54 (O(CH₂)₂CH₂CH₂), 1.62-1.69 (m, 2H, OCH₂CH₂(CH₂)₂), 1.72-1.82 (m, 1H, O(CH₂)₃CHH), 2.19-2.23 (m, 1H, O(CH₂)₃CHH), 2.85-2.98 (m, 3H, ArCH₂CHH), 3.02-3.07 (m, 1H, ArCH₂CHH), 3.13-3.21 (m, 2H, CHCH₂Ar), 3.89-3.93 (m, 1H, OCHH(CH₂)₃), 4.00-4.05 (m, 1H, OCHH(CH₂)₃), 4.61-4.62 (m, 1H, NHCHCO), 4.78 (q, J=6.6 Hz, 1H, NHCHCHO), 6.33-6.35 (m, 1H, pyrrole H), 6.42 (br s, 2H, OArH), 6.43 (d, J=7.2 Hz, 1H, NHCHCHO), 6.46 (d, J=7.8 Hz, 1H, NHCHCO), 6.68 (br s, 2H, OArH), 6.71-6.73 (m, 1H, pyrrole H), 7.09 (d, J=9.0 Hz, 1H, ArH), 7.14 (d, J=7.2 Hz, 1H, ArH), 7.20-7.28 (m, 3H, ArH), 8.47 (br d, J=13.2 Hz, 1H, pyrrole NH), 9.66 (s, 1H, CHO); ¹³C NMR (CDCl₃, 150 MHz) δ 19.6, 27.5, 31.1, 34.0, 35.2, 42.3, 59.8, 65.6, 109.9, 115.9, 127.6, 128.9, 129.0, 129.1. 129.3, 129.4, 131.7, 135.0, 135.2, 157.2, 159.4, 171.3, 192.2, 198.2; HRMS (ES) 502.2325 (MH⁺); C₂₉H₃₂N₃O₅ requires 502.2336; [α]_{D}=+2.0 (c 0.1 in (CH₃)₂SO).

XXX 3a

Macrocyclic ester 18 (40 mg, 0.08 mmol) was hydrolized (General Procedure G) to the carboxylic acid, which was coupled to (L)-Leucinol (General Procedure D) and the crude product was purified by flash chromatography on silica using a gradient of ethtyl acetate and (50/70) petroleum ether to give 3a (25 mg, 54%) as a white solid. mp 122-125° C.; ¹H NMR (CDCl₃, 600 MHz) δ 0.84, (d, J=3.3 Hz, 3H, CH₂CH(CH₃)₂), 0.85 (d, J=3.3 Hz, 3H, CH₂CH(CH₃)₂), 1.30-1.38 (m, 2H, CH₂CH(CH₃)₂), 1.53-1.58 (m, 1H, CH₂CH(CH₃)₂), 1.92-1.95 (m, 4H, OCH₂CH₂CH₂CH₂O), 2.67-2.70 (m, 1H, ArCH₂CHH), 2.91-3.04 (m, 3H, ArCH₂CH₂, CHCHHAr), 3.13-3.18 (m, 1H, ArCH₂CHH), 3.34 (dd, J=14.4 and 4.8 Hz, 1H, CHCHHAr), 3.50 (br s, 1H, CH₂OH), 3.72 (d, J=9.9 Hz, 1H, CHHOH), 3.82 (d, J=9.9 Hz, 1H, CHHOH), 3.85-3.98 (m, 4H, OCH₂(CH₂)₂CH₂O), 4.08-4.14 (m, 1H, CHCH₂OH), 4.98 (q, J=7.2 Hz, 1H, CHCH₂Ar), 6.11-6.12 (m, 1H, pyrrole H), 6.16-6.17 (m, 1H, pyrrole H), 6.45 (d, J=7.5 Hz, 1H, NHCHCO), 6.65 (d, J=8.4 Hz, 2H, OArH), 6.73 (d, J=8.1 Hz, 2H, OArH), 6.91 (d, J=8.4 Hz, 2H, OArH), 7.01 (d, J=8.1 Hz, 2H, OArH), 7.40 (d, J=9.0 Hz, 1H, NHCHCH₂OH), 10.53 (br s, 1H, pyrrole NH); ¹³C NMR (CDCl3, 150 MHz) δ 22.6, 22.9, 25.1, 25.4, 25.5, 32.8, 37.2, 40.3, 40.4, 49.6, 54.0, 65.4, 67.2, 67.5, 110.3, 114.4, 114.8, 117.9, 128.1, 129.7, 130.3, 130.6, 132.1, 134.6, 157.5, 158.1, 160.1, 171.3, 192.8; HRMS (ES) 576.30682 (MH⁺); C₃₃H₄₂N₃O₆ requires 576.30681; [α]_{D}=+4.0 (c 0.1 in (CH₃)₂SO).

XXX 3b

Macrocyclic ester 18 (40 mg, 0.08 mmol) was hydrolized (General Procedure G) to the carboxylic acid, which was coupled to (L)-Phenalaninol (General Procedure D) and the crude product was purified by flash chromatography on silica using a gradient of ethtyl acetate and (50/70) petroleum ether to give 3b (28 mg, 55%) as a white solid. mp 118-120° C.; ¹H NMR (CDCl₃, 600 MHz) δ 1.92-1.99 (m, 4H, OCH₂CH₂CH₂CH₂O), 2.70-2.73 (m, 1H, ArCH₂CHH), 2.84 (d, J=7.8 Hz, 2H, CHCH₂Ar), 2.95-3.05 (m, 3H, ArCH₂CH₂, CHCHHArO), 3.17-3.21 (m, 1H, ArCH₂CHH), 3.34 (dd, J=14.4 and 6 Hz, 1H, CHCHHArO), 3.76-3.85 (m, 3H, CH₂OH), 3.90-4.02 (m, 4H, OCH₂(CH₂)₂CH₂O), 4.22-4.26 (m, 1H, CHCH₂Ar), 5.15 (q, J=7 Hz, 1H, CHCH₂ArO), 6.07-6.08, 6.14-6.15 (m, 2H, 2×pyrrole H), 6.32 (d, J=7 Hz, 1H, NHCHCO), 6.66 (d, J=8.4 Hz, 2H, OArH), 6.73 (d, J=8.4 Hz, 2H, OArH), 6.92 (d, J=8.4 Hz, 2H, OArH), 6.97 (d, J=8.4 Hz, 2H, OArH), 7.10-7.12 (m, 1H, ArH), 7.16-7.19 (m, 4H, ArH), 7.78 (d, J=7.8 Hz, 1H, NHCHCH₂OH), 10.78 (br s, 1H, pyrrole NH); ¹³C NMR (CDCl₃, 150 MHz) δ 25.4, 25.5, 33.1, 37.3, 37.4, 40.7, 52.7, 53.5, 63.1, 67.2, 67.6, 110.2, 114.3, 114.7, 118.4, 126.5, 127.9, 128.6, 129.5, 129.8, 130.5, 130.8, 132.2, 134.6, 138.1, 157.6, 158.0, 159.9, 171.3, 193.2; HRMS (ES) 610.29114 (MH⁺); C₃₆H₄₀N₃O₆ requires 610.29116; [α]_{D}=+1.4 (c 0.2 in (CH₃)₂SO).

XXX 3c

Alcohol 3a (24 mg, 0.042 mmol) was oxidized (General Procedure H) and the crude product was purified by rp-HPLC to give 3c (10 mg, 41%) as a white oil. ¹H NMR (CDCl₃, 600 MHz) δ 0.84, (t, J=5.7 Hz, 6H, CH₂CH(CH₃)₂), 1.43-1.51 (m, 1H, CHHCH(CH₃)₂), 1.66-1.74 (m, 2H, CHHCH(CH₃)₂), 1.93 (br s, 4H, OCH₂CH₂CH₂CH₂O), 2.68-2.71 (m, 1H, ArCH₂CHH), 2.94-3.07 (m, 3H, ArCH₂CH₂, CHCHHAr), 3.09-3.13 (m, 1H, ArCH₂CHH), 3.33 (dd, J=14.4 and 4.8 Hz, 1H, CHCHHAr), 3.81-3.97 (m, 4H, OCH₂(CH₂)₂CH₂O), 4.52-4.60 (m, 1H, NHCHCHO), 4.74-4.79 (m, 1H, NHCHCO), 6.07-6.09 (m, 1H, pyrrole H), 6.14-6.15 (m, 1H, pyrrole H), 6.24 (d, J=6.0 Hz, 1H, NHCHCHO), 6.63 (d, J=8.7 Hz, 2H, OArH), 6.74 (d, J=9.0 Hz, 2H, OArH), 6.91 (d, J=8.7 Hz, 2H, OArH), 7.01 (d, J=9.0 Hz, 2H, OArH), 7.05 (d, J=7.2 Hz, 1H, NHCHCO), 9.61 (s, 1H, CHO), 10.02 (br s, 1H, pyrrole NH); ¹³C NMR (CDCl₃, 150 MHz) δ 22.0, 23.1, 24.9, 25.3, 25.3, 32.8, 36.3, 37.8, 40.3, 54.3, 57.6, 67.1, 67.3, 110.6, 114.4, 114.6, 117.2, 127.8, 129.0, 129.7, 130.3, 132.2, 134.9, 157.5, 158.2, 160.4, 171.5, 192.3, 199.2; HRMS (ES) 574.2900 (MH⁺); C₃₃H₄₀N₃O₆ requires 574.2912; [α]_{D}=+3.5 (c 0.1 in (CH₃)₂SO).

XXX 3d

Alcohol 3b (18 mg, 0.03 mmol) was oxidized (General Procedure H) and the crude product was purified by rp-HPLC to give 3d (12 mg, 65%) as a white oil. ¹H NMR (CDCl₃, 600 MHz) δ 1.92 (br s, 4H, OCH₂CH₂CH₂CH₂O), 2.68-2.70 (m, 1H, ArCH₂CHH), 2.93 (dd, J=14.7 and 10.5 Hz, 1H CHCHHArO), 2.96-3.04 (m, 2H, ArCH₂CH₂), 3.06-3.14 (m, 2H, CHCHHAr, ArCH₂CHH), 3.17 (dd, J=14.3 and 6.3 Hz, 1H, CHCHHAr), 3.26 (dd, J=14.7 and 4.5 Hz, 1H, CHCHHArO), 3.79-3.97 (m, 4H, OCH₂(CH₂)₂CH₂O), 4.67-4.71 (m, 1H, NHCHCO), 4.74 (q, J=6.8 Hz, 1H, CHCH₂Ar), 6.00-6.02, 6.14-6.15 (m, 2H, NHCHCHO, pyrrole H), 6.11-6.12 (m, 1H, pyrrole H), 6.62 (d, J=8.4 Hz, 2H, OArH), 6.74 (d, J=8.4 Hz, 2H, OArH), 6.90 (d, J=8.4 Hz, 2H, OArH), 6.98 (d, J=8.4 Hz, 2H, OArH), 7.08-7.13 (m, 6H, ArH, NHCHCHO), 9.67 (s, 1H, CHO), 9.89 (br s, 1H, pyrrole NH); ¹³C NMR (CDCl₃, 150 MHz) δ 25.3, 32.8, 35.1, 35.8, 40.3, 54.1, 59.9, 67.1, 67.3, 110.3, 114.4, 115.1, 117.1, 127.3, 127.8, 128.8, 129.0, 129.4, 129.7, 130.3, 132.2, 135.4, 157.5, 158.2, 160.4, 171.3, 192.3, 198.5; HRMS (ES) 608.2736 (MH⁺); C₃₆H₃₈N₃O₆ requires 608.2755; [α]D=+1.1 (c 0.2 in (CH₃)₂SO).

### Methyl 3-(4-allyloxyphenyl)propanoate 5

To a solution of methyl 3-(4-hydroxyphenyl)propanoate, 4 (9.05 g, 50.2 mmol) in DMF (66 mL) was added sequentially K₂CO₃ (13.9 g, 100 mmol), tetrabutylammonium iodide (1.89 g, 5.0 mmol) and allyl bromide (7.59 g, 63 mmol). The resulting solution was stirred at ambient temperature under a nitrogen atmosphere for 18 h and was poured into ice-water. The mixture was extracted with ethyl acetate (4×). The combined organic extracts were washed with 1M aqueous HCl, H₂O (2×), brine, dried over Na₂SO₄ and volatiles were removed in vacuo to afford 5 in quantitative yield (11.01 g) as a yellow oil. ¹H NMR (CDCl₃, 300 MHz) δ 2.60 (t, J=7.8 Hz, 2H, ArCH₂CH₂), 2.89 (t, J=7.8 Hz, 2H, ArCH₂CH₂), 3.66 (s, 3H, OCH₃), 4.50-4.52 (m, 2H, OCH₂CHCH₂), 5.25-5.30 (m, 1H, OCH₂CHCHH), 5.37-5.44 (m, 1H, OCH₂CHCHH), 5.99-6.12 (m, 1H, OCH₂CHCH₂), 6.84 (d, J=8.7 Hz, 2H, OArH), 7.11 (d, J=8.7 Hz, 2H, OArH); ¹³C NMR (CDCl₃, 75 MHz) δ 30.1, 35.9, 51.6, 68.8, 114.7, 117.6, 129.2, 132.7, 133.4, 157.1, 173.4.

### 3-(4-Allyloxyphenyl)propanoic acid 6

Lithium hydroxide (6.34 g, 246 mmol) was added to methyl 3-(4-allyloxyphenyl)propionate, 5 (11.01 g, 50.0 mmol) in 3:1 THF/H₂O (110 mL) and stirred at 40° C. for 3.5 h. The reaction mixture was cooled to 0° C. and acidified to pH 1 with 2M aqueous HCl. The resulting mixture was extracted ethyl acetate (3×). The combined organic extracts were washed with water (2×) and brine, dried over Na₂SO₄ and volatiles were removed in vacuo to yield carboxylic acid 6 in quantitative yield (10.31 g) as a yellow solid. m.p. 87-89° C., lit. m.p. 89-90° C. {5}; ¹H NMR (DMSO-d₆, 300 MHz) δ 2.45-2.50 (m, 2H, ArCH₂CH₂), 2.74 (t, J=7.7 Hz, 2H, ArCH₂CH₂), 4.50-4.53 (m, 2H, OCH₂CHCH₂), 5.22-5.26 (m, 1H, OCH₂CHCHH), 5.34-5.41 (m, 1H, OCH₂CHCHH), 5.96-6.09 (m, 1H, OCH₂CHCH₂), 6.84 (d, J=8.6 Hz, 2H, OArH), 7.12 (d, J=8.6 Hz, 2H, OArH), 12.11 (br s, 1H, OH); ¹³C NMR (DMSO-d₆, 75 MHz) δ 29.7, 35.8, 68.3, 114.7, 117.5, 129.4, 133.1, 134.1, 156.7, 174.0.

### 3-(4-Allyloxyphenyl)propanoyl chloride 7

Carboxylic acid 6 (1.669 g, 8.0 mmol) in dry dichloromethane (26 mL) at 0° C. was reacted with thionyl chloride (3.98 mL, 48.0 mmol). The solution was stirred for 10 min to at 0° C., heated to 40° C. and stirred for 18 h. Volatiles were removed in vacuo to afford acid chloride 7 in quantitative yield (1.817 g) as a dark yellow oil. ¹H NMR (CDCl₃, 300 MHz) δ 2.96 (t, J=7.4 Hz, 2H, ArCH₂CH₂), 3.18 (t, J=7.4 Hz, 2H, ArCH₂CH₂), 4.52-4.55 (m, 2H, OCH₂CHCH₂), 5.28-5.33 (m, 1H, OCH₂CHCHH), 5.39-5.46 (m, 1H, OCH₂CHCHH), 6.00-6.13 (m, 1H, OCH₂CHCH₂), 6.88 (d, J=8.6 Hz, 2H, OArH), 7.12 (d, J=8.6 Hz, 2H, OArH); ¹³C NMR (CDCl₃, 75 MHz) δ 30.1, 48.7, 68.7, 114.9, 117.5, 129.2, 130.7, 133.2, 157.4, 173.0.

### 2-Trichloroacetyl-1H-pyrrole 9

Pyrrole (6.72 g, 100 mmol) in ether (15 mL) was added drop wise over a 1 hr period to a solution of trichloroacetyl chloride (20.0 g, 110 mmol) in ether (50 mL). The resulting mixture was stirred for 3 h before the reaction was quenched slowly with the addition of potassium carbonate (8.97 g) in water (35 mL). The layers were separated, and the organic phase was dried over MgSO₄. Volatiles were removed in vacuo to obtain a grey crystalline solid, which was recrystallised from n-hexane (350 mL) by addition of silica gel 60 (6 g). The hot mixture was filtered and the filtrate was allowed to cool to −12° C. The white precipitate was collected by filtration and the volume of the filtrate was reduced (˜50 mL) in vacuo. The filtrate was cooled to 4° C. and the grey crystalline solid was collected. All precipitates were dried in vacuo at ambient temperature to give compound 1 (17.8 g, 84%) as a white crystalline solid. mp 71-73° C., lit. mp 72-74° C. {6}; ¹H NMR (CDCl₃, 300 MHz) δ 6.37-6.40 (m, 1H, pyrrole H4), 7.18-7.20 (m, 1H, pyrrole H3), 7.39-7.42 (m, 1H, pyrrole H5), 9.79 (br s, 1H, NH). ¹³C NMR (CDCl₃, 75 MHz) δ 94.9, 111.8, 121.3, 122.8, 127.3, 173.2. Experimental data as per literature. {6}

### Ethyl 1H-pyrrole-2-carboxylate 10

Sodium metal (0.15 g, 6.5 mmol) was added to a solution of anhydrous ethanol (20 mL) and once the sodium had dissolved, pyrrole 1 (10.45 g, 49.2 mmol) was added portion wise over a period of 10 mins. Upon completion, the mixture was stirred for 40 mins at ambient temperature and volatiles were removed in vacuo to give a dark red oil, which was redissolved in diethyl ether (30 mL) and extracted with 3 M aqueous hydrochloric acid (6 mL). The ether phase was separated, and the aqueous phase was further extracted with diethyl ether (20 mL). The ether phases were combined, washed with saturated NaHCO₃ (5 mL), and dried over MgSO₄. Volatiles were removed in vacuo to obtain a brown oil, which crystallised upon standing to form tan crystals (6.562 g). The tan crystals was purified by distillation under reduced pressure (130° C., 5 mm Hg), to give compound 10 (6.213 g, 91%) as a colourless oil which crystallised upon standing to form white crystals. m.p. 36-37° C., lit. mp 38-39.5° C. {6}; ¹H NMR (CDCl₃, 600 MHz) δ 1.36 (t, ³J=7.2 Hz, 3H, OCH₂CH₃), 4.33 (q, ³J=7.2 Hz, 2H, OCH₂CH₃), 6.25-6.27 (m, 1H, pyrrole H4), 6.91-6.93 (m, 1H, pyrrole H3), 6.94-6.95 (m, 1H, pyrrole H5), 9.31 (br s, 1H, NH). ¹³C NMR (CDCl₃, 150 MHz) δ 14.43, 60.29, 110.35, 115.07, 122.70, 122.99, 161.25. Experimental data as per literature. {6}

### Ethyl 5-undec-10-enoyl-1H-pyrrole-2-carboxylate 11a

Ethyl 1H-pyrrole-2-carboxylate 10 (505 mg, 3.6 mmol) was coupled to 10-undecenoyl chloride (1.6 mL, 7.5 mmol) (General Procedure A) and the crude product was purified by flash chromatography (petroleum ether/ethyl acetate 9:1) to afford the compound 11a (588 mg, 53%) as a yellow oil, which crystallised upon standing. m.p. 41-43° C.; ¹H NMR (CDCl₃, 600 MHz) δ 1.25-1.39 (m, 13H, (CH₂)₂(CH₂)₅CH₂CHCH₂, OCH₂CH₃), 1.72 (quin, J=7.5 Hz, 2H, CH₂CH₂(CH₂)₆CHCH₂), 2.04 (q, J=7.2 Hz, 2H, (CH₂)₂(CH₂)₅CH₂CHCH₂), 2.79 (t, J=7.8 Hz, 2H, COCH₂), 4.36 (q, J=7.2 Hz, 2H, OCH₂CH₃), 4.93 (d, J=10.2 Hz, 1H, (CH₂)₈CHCHH), 4.99 (d, J=17.4 Hz, 1H, (CH₂)₈CHCHH), 5.78-5.84 (m, 1H, (CH₂)₈CHCH₂), 6.83-6.83 (m, 1H, pyrrole H), 6.88-6.89 (m, 1H, pyrrole H), 9.78 (br s, 1H, pyrrole NH); ¹³C NMR (CDCl₃, 150 MHz) δ 14.5, 24.9, 29.0, 29.2, 29.4, 29.5, 33.9, 38.6, 61.3, 114.3, 115.5, 115.6, 127.2, 134.1, 139.3, 160.5, 191.8; HRMS (ES) 306.2052 (MH⁺); C₁₈H₂₈NO₃ requires 306.2064.

### 5-Undec-10-enoyl-1H-pyrrole-2-carboxylic acid 11b

Ester 11a (100 mg, 0.33 mmol) was hydrolysed (General Procedure B) to afford carboxylic acid 12b (77 mg, 85%) as a pale yellow solid. m.p. 113-115° C.; ¹H NMR (CDCl₃, 300 MHz) δ 1.24-1.42 (m, 10H, (CH₂)₂(CH₂)₅CH₂CHCH₂), 1.69-1.77 (m, 2H, CH₂CH₂(CH₂)₅CH₂CHCH₂), 2.01-2.07 (m, 2H, (CH₂)₂(CH₂)₅CH₂CHCH₂), 2.87 (t, J=7.5 Hz, 2H, COCH₂), 4.92-5.02 (m, 2H, (CH₂)₈CHCH₂), 5.75-5.88 (m, 1H, (CH₂)₈CHCH₂), 6.95-7.04 (m, 2H, pyrrole H), 11.18 (br s, 1H, pyrrole NH); ¹³C NMR (CDCl₃, 75 MHz) δ 25.0, 29.0, 29.2, 29.4, 29.5, 33.9, 38.9, 114.3, 117.2, 117.4, 128.0, 134.6, 139.3, 164.2, 193.5; HRMS (ES) 278.1740 (MH⁺); C₁₆H₂₄NO₃ requires 278.1751.

### Ethyl 5-[3-(4-allyloxyphenyl)propanoyl]-1H-pyrrole-2-carboxylate 12a

Ethyl 1H-pyrrole-2-carboxylate 10 (2.23 g, 16.0 mmol) was coupled to acid chloride 7 (7.19 g, 32.0 mmol) (General Procedure A) and the crude product was purified by flash chromatography (petroleum ether/ethyl acetate 4:1) to afford the compound 12a (2.076 g, 40%) as a yellow oil, which crystallised upon standing. m.p. 69-73° C.; ¹H NMR (CDCl₃, 300 MHz) δ 1.37 (t, J=7.2 Hz, 3H, OCH₂CH₃), 2.96-3.01 (m, 2H, ArCH₂CH₂), 3.07-3.13 (m, 2H, ArCH₂CH₂), 4.36 (q, J=7.2 Hz, 2H, OCH₂CH₃), 4.50-4.52 (m, 2H, OCH₂CHCH₂), 5.26-5.30 (m, 1H, OCH₂CHCHH), 5.36-5.44 (m, 1H, OCH₂CHCHH), 5.98-6.11 (m, 1H, OCH₂CHCH₂), 6.80-6.88 (m, 4H, pyrrole H, OArH), 7.13 (d, J=8.7 Hz, 2H, OArH), 9.83 (br s, 1H, NH); ¹³C NMR (CDCl₃, 50 MHz) δ 14.5, 29.6, 40.5, 61.3, 69.0, 114.9, 115.6, 117.7, 127.3, 129.4, 133.1, 133.5, 133.9, 157.2, 160.5, 190.6; HRMS (ES) 328.1541 (MH⁺); C₁₉H₂₂NO₄ requires 328.1549.

### 5-[3-(4-allyloxyphenyl)propanoyl]-1H-pyrrole-2-carboxylic acid 12b

Ester 12a (584 mg, 1.8 mmol) was hydrolysed (General Procedure B) to afford carboxylic acid 12b (511 mg, 96%) as a yellow solid. m.p. 169-172° C.; ¹H NMR (CDCl₃, 300 MHz) δ 3.01 (t, J=6.9 Hz, 2H, ArCH₂CH₂), 3.16 (t, J=7.2 Hz, 2H, ArCH₂CH₂), 4.51 (d, J=5.1 Hz, 2H, OCH₂CHCH₂), 5.28 (d, J=10.2 Hz, 1H, OCH₂CHCHH), 5.40 (d, J=17.1 Hz, 1H, OCH₂CHCHH), 5.99-6.11 (m, 1H, OCH₂CHCH₂), 6.85 (d, J=8.6 Hz, 2H, OArH), 6.92 (br 1H, pyrrole H), 7.01 (br, 1H, pyrrole H), 7.13 (d, J=8.6 Hz, 2H, OArH), 10.99 (br s, 1H, NH), COOH was not observed; ¹³C NMR (CDCl₃, 75 MHz) δ 29.6, 40.7, 68.8, 114.9, 117.1, 117.4, 117.7, 127.3, 129.4, 133.1, 133.5, 133.9, 157.2, 164.1, 192.1; HRMS: (ES) 300.1241 (MH⁺) C₁₇H₁₈NO₄ requires 300.1236.

### (S)-methyl 2-(5-undec-10-enoyl-1H-pyrrole-2-carboxamido)pent-4-enoate 13

Carboxylic acid 11b (419 mg, 1.5 mmol) was coupled to (S)-allyl-Gly-OMe (314 mg, 1.8 mmol) (General Procedure C) and the crude product was purified by flash chromatography (petroleum ether/ethyl acetate 4:1) to afford the acyclic diene 14 (280 mg, 48%) as a pale yellow oil. ¹H NMR (CDCl₃, 600 MHz) δ 1.26-1.37 (m, 10H, (CH₂)₂(CH₂)₅CH₂CHCH₂), 1.68-1.73 (m, 2H, CH₂CH₂(CH₂)₅CH₂CHCH₂), 2.03 (q, J=7.0 Hz, 2H, (CH₂)₂(CH₂)₅CH₂CHCH₂), 2.57-2.63 (m, 1H, NHCHCHH), 2.67-2.72 (m, 1H, NHCHCHH), 2.77 (t, J=7.2 Hz, 2H, COCH₂), 3.79 (s, 3H, OCH₃), 4.86 (q, J=6.4 Hz, 1H, NHCH), 4.93 (d, J=10.2 Hz, 1H, (CH₂)₈CHCHH), 4.99 (d, J=18.0 Hz, 1H, (CH₂)₈CHCHH), 5.15 (d, J=13.5 Hz, 2H, CHCH₂CHCH₂), 5.65-5.76 (m, 1H, CHCH₂CHCH₂), 5.77-5.84 (m, 1H, (CH₂)₈CHCH₂), 6.57 (br d, J=7.8 Hz, 1H, NHCH), 6.60 (br s, 1H, pyrrole H), 6.83 (br s, 1H, pyrrole H), 9.97 (br s, 1H, pyrrole NH); ¹³C NMR (CDCl₃, 150 MHz) δ 24.9, 29.0, 29.2, 29.4, 29.5, 33.9, 36.8, 38.5, 51.8, 52.7, 110.3, 114.3, 115.5, 119.7, 129.6, 132.1, 133.7, 139.3, 159.6, 172.2, 191.5; FIRMS (ES) 389.2427 (MH⁺); C₂₂H₃₃N₂O₄ requires 389.2435.

### Methyl (2S)-2-[[5-[3-(4-allyloxyphenyl)propanoyl]-1H-pyrrole-2-carbonyl]amino]pent-4-enoate 14

Carboxylic acid 12b (480 mg, 1.6 mmol) was coupled to (S)-allyl-Gly-OMe (297 mg, 1.8 mmol) (General Procedure D) and the crude product was purified by flash chromatography (petroleum ether/ethyl acetate 1:1) to afford the acyclic diene 14 (621 mg, 94%) as a dark yellow oil. ¹H NMR (CDCl₃, 300 MHz) δ 2.58-2.72 (m, 2H, CHCH₂CHCH₂), 2.95-3.00 (m, 2H, ArCH₂CH₂), 3.05-3.11 (m, 2H, ArCH₂CH₂), 3.79 (s, 3H, OCH₃), 4.51 (dt, J=5.1 and 1.5 Hz, 2H, OCH₂CHCH₂), 4.85 (dt, J=7.7 and 5.6 Hz, 1H, CHCH₂CHCH₂), 5.12-5.18 (m, 2H, CHCH₂CHCH₂), 5.28 (dq, J=10.4 and 1.6 Hz, 1H, OCH₂CHCHH) 5.40 (dq, J=17.4 and 1.6 Hz, 1H, OCH₂CHCHH), 5.65-5.79 (m, 1H, CHCH₂CHCH₂), 5.99-6.11 (m, 1H, OCHCH₂CHCH₂), 6.53 (d, J=7.7 Hz, 1H, NHCH), 6.57 (dd, J=4.0 and 2.7 Hz, 1H, pyrrole H), 6.80 (dd, J=4.0 and 2.7 Hz, 1H, pyrrole H), 6.84 (dt, J=8.7 and 2.3 Hz, 2H, OArH), 7.13 (dt, J=8.7 and 2.3 Hz, 2H, OArH), 9.94 (br s, 1H, pyrrole NH); ¹³C NMR (CDCl₃, 75 MHz) 8 ¹³C NMR (CDCl₃, 75 MHz) δ 29.5, 36.6, 40.3, 51.6, 52.6, 68.8, 110.1, 113.1, 114.9, 117.6, 119.4, 129.3, 129.4, 130.9, 132.2, 133.4, 133.5, 157.1, 159.7, 172.4, 190.3; HSMS: (ES) 411.1909 (MH⁺) C₂₃H₂₇N₂O₅ requires 411.1920.

### Methyl (2S)-3-(4-allyloxyphenyl)-2-[[5-[3-(4-allyloxyphenyl)propanoyl]-1H-pyrrole-2-carbonyl]amino]propanoate 15

Carboxylic acid 12b (199 mg, 0.66 mmol) was coupled to (S)-allyl-Try-OMe (207 mg, 0.76 mmol) (General Procedure C) and the crude product was purified by flash chromatography (petroleum ether/ethyl acetate 1:1) to afford the acyclic diene 15 (323 mg, 94%) as a yellowish brown oil. ¹H NMR (CDCl₃, 300 MHz) δ 2.94-2.99 (m, 2H, ArCH₂CH₂), 3.04-3.10 (m, 2H, ArCH₂CH₂), 3.15 (dd, J=5.1 and 5.1 Hz, 2H, NHCH), 3.76 (s, 3H, OCH₃), 4.48-4.52 (m, 4H, 2×OCH₂CHCH₂), 5.02 (dt, J=7.8 and 5.7 Hz, 1H, NHCH), 5.25-5.30 (m, 2H, 2×OCH₂CHCHH) 5.36-5.43 (m, 2H, 2×OCH₂CHCHH), 5.97-6.11 (m, 2H, 2×OCH₂CHCH₂), 6.50 (dd, J=4.1 and 2.6 Hz, 1H, pyrrole H), 6.59 (d, J=7.8 Hz, 1H, NHCH), 6.77 (dd, J=4.1 and 2.6 Hz, 1H, pyrrole H), 6.82 (d, J=8.7 Hz, 2H, OArH), 6.84 (d, J=8.7 Hz, 2H, OArH), 7.02 (d, J=8.7 Hz, 2H, OArH), 7.13 (d, J=8.7 Hz, 2H, OArH), 10.07 (br s, 1H, pyrrole NH); ¹³C NMR (CDCl₃, 75 MHz) δ 29.6, 37.2, 40.4, 52.7, 53.4, 68.9, 69.0, 110.4, 114.9, 115.0, 115.7, 117.8, 117.9, 127.7, 129.4, 129.8, 130.4, 133.3, 133.5, 157.2, 157.9, 159.4, 172.1, 190.3; HRMS (ES) 517.2321 (MH⁺) C₃₀H₃₃N₂O₆ requires 517.2339.

### (S)-methyl 2,16-dioxo-3,20-diazabicyclo[15.2.1]icosa-1(19),17-diene-4-carboxylate 16

RCM of acyclic diene 13 (308 mg, 0.79 mmol) (General Procedure E) gave a mixture of cis and trans macrocycles (100 mg, 35%). The crude mixture was hydrogenated (General Procedure F) and purified by flash chromatography on silica using a gradient of ethyl acetate and (50/70) petroleum ether to give 16 (79 mg, 79%) as a white oil. ¹H NMR (CDCl₃, 600 MHz) δ 0.80-0.94 (m, 2H, CO(CH₂)₈CH₂), 0.96-1.13 (m, 2H, CO(CH₂)₃CH₂), 1.18-1.38 (m, 12H, CO(CH₂)₂CH₂CH₂(CH₂)₄CH₂CH₂CH₂), 1.68-1.75 (m, 1H, CO(CH₂)₁₀CHH), 1.78-1.88 (m, 2H, COCH₂CH₂), 2.17-2.25 (m, 1H, CO(CH₂)₁₀CHH), 2.60 (br s, 1H, COCHH), 2.92 (br s, 1H, COCHH), 3.80 (s, 3H, OCH₃), 4.93 (dt, J=8.0 and 3.9 Hz, 1H, NHCH₃), 6.66 (br s, 1H, NHCH), 6.71 (s, 1H, pyrrole H), 6.91-6.92 (m, 1H, pyrrole H), 10.19 (br s, 1H, pyrrole NH); ¹³C NMR (CDCl₃, 150 MHz) δ 24.0, 26.7, 27.7, 28.4, 29.0, 29.3, 29.4, 29.7, 30.1, 39.0, 52.6, 52.7, 110.8, 116.5, 130.2, 134.1, 159.5, 173.0, 194.1; HRMS (ES) 363.2274 (MH⁺); C₂₀H₃₁N₂O₄ requires 363.2278.

XXX 17

RCM of acyclic diene 14 (209 mg, 0.51 mmol) (General Procedure E) gave a mixture of cis and trans macrocycles (103 mg, 53%). The crude mixture was hydrogenated (General Procedure F) purified by flash chromatography on silica using a gradient of ethyl acetate and (50/70) petroleum ether, and recrystallized from ethyl acetate to give 17 (69 mg, 67%) as a white needle-like crystals. mp 198-200° C.; ¹H NMR (CDCl₃, 600 MHz) δ 1.33-1.40 (m, 1H, O(CH₂)₂CHHCH₂), 1.45-1.52 (m, 1H, O(CH₂)₂CHHCH₂), 1.64-1.70 (m, 1H, OCH₂CHH(CH₂)₂), 1.72-1.81 (m, 2H, OCH₂CHHCH₂CHH), 2.31-2.37 (m, 1H, OCH₂CH₂CH₂CHH), 2.83-2.95 (m, 3H, ArCHHCH₂), 3.04-3.08 (m, 1H, ArCHHCH₂), 3.80 (s, 3H, OCH₃), 3.89-3.92 (m, 1H, OCHH(CH₂)₃), 4.03-4.06 (m, 1H, OCHH(CH₂)₃), 4.75-4.78 (m, 1H, NHCH), 6.37-6.40 (m, 2H, pyrrole H, OArH), 6.50 (d, J=3.6 Hz, 1H, NHCH), 6.61 (br s, 1H, OArH), 6.77 (br s, 1H, pyrrole H), 6.81 (br s, 1H, OArH), 7.18 (br s, 1H, OArH), 8.34 (br s, 1H, pyrrole NH); ¹³C NMR (CDCl₃, 150 MHz, 0° C.) δ 19.1, 27.4, 29.5, 33.9, 42.5, 52.0, 52.9, 65.2, 109.5, 115.7, 129.2, 131.8, 135.0, 157.4, 158.8, 172.6, 192.0; HRMS (ES) 385.1778 (MH⁺); C₂₁H₂₅N₂O₅ requires 385.1758.

XXX 18

RCM of acyclic diene 15 (150 mg, 0.29 mmol) (General Procedure E) gave a mixture of cis and trans macrocycles (88 mg, 62%). The crude mixture was hydrogenated (General Procedure F) and purified by flash chromatography on silica using a gradient of ethyl acetate and (50/70) petroleum ether to give 18 (80 mg, 91%) as a yellow oil. ¹H NMR (CDCl₃, 600 MHz) δ 1.88-1.98 (m, 4H, OCH₂CH₂CH₂CH₂O), 2.67-2.71 (m, 1H, ArCHHCH₂), 2.93-3.03 (m, 3H, ArCH₂CH₂, NHCHCHH), 3.11 (dt, J=12.0 and 5.0 Hz, 1H, ArCHHCH₂), 3.42 (dd, J=14.1 and 5.0 Hz, 1H, NHCHCHH), 3.84 (s, 3H, OCH₃), 3.88 (t, J=5.7 Hz, 2H, (CH₂)₂ArOCH₂), 3.95-4.04 (m, 2H, CH₂OArCH₂CH), 4.85 (q, J=7.2 Hz, 1H, NHCH), 6.08-6.12 (m, 2H, pyrrole H), 6.16 (d, J=7.2 Hz, 1H, NHCH), 6.64 (d, J=8.7 Hz, 2H, OArH), 6.72 (d, J=8.7 Hz, 2H, OArH), 6.88-6.92 (m, 4H, OArH), 9.71 (br s, 1H, pyrrole NH); ¹³C NMR (CDCl3, 150 MHz) δ 25.4, 25.5, 32.9, 36.2, 40.3, 52.8, 53.2, 67.2, 67.6, 110.1, 114.2, 114.9, 117.0, 127.2, 129.8, 129.9, 130.4, 132.3, 134.5, 157.5, 158.1, 159.5, 172.2, 192.1; HRMS (ES) 491.2195 (MH⁺); C₂₈H₃₂N₂O₆ requires 491.2177.

### REFERENCES

{1} Thomson V. F.; Saldaña, S.; Cong, J.; Goll, D. E. Anal. Biochem. 2000, 279, 170-178. {2} Morrison, J. F. Trends Biochem. Sci. 1982, 7, 102-105. {3} Liljeblad, A.; Kanerva, L. T. Tetrahedron 2006, 62, 5831. {4} Dixon, M. Biochem. J. 1953, 55, 170. {5} Stoessl, A. Tetrahedron Lett. 1966, 7, 2287-2292. {6} Wallace, D. M.; Leung, S. H.; Senge, M. O.; Smith, K. M. J. Org. Chem. 1993, 58, 7245-7251.

### Example 3

### Synthesis and Biological Evaluation of 18- and 24-Membered Macrocycles as Inhibitors for Calpain

The present study is based on extended macrocycles, which are less peptide-like and designed to adopt a β-strand geometry, a conformation universally adopted by inhibitors and substrates upon binding to a protease.

Structural studies have shown that 17- and 18-membered ring systems were most favourable and incorporation of a phenyl group at the P3 position favours binding while also assisting in adoption of β-strand conformation. In this study, we eliminated this by addition of an aromatic group in the peptide backbone, allowing the investigation of side chain flexibility and polarity.

Consequently, we synthesised a series of 18- and 24-membered macrocycles, constraining the P2-P4 amino acid by ring closing metathesis (RCM) with incorporation of a pyrrole group in place of a P3 amino acid residue, thereby reducing the peptide-like nature, but still maintaining the required β-strand geometry of the backbone. The polarity/aromaticity of the P2 and P4 side chains were varied, by incorporating phenyl groups, which would also affect the flexibility of the macrocyclic side chains. The macrocycle is extended in the C-terminal end, to incorporate the P1 residue, which is used to incorporate selectivity for a particular enzyme. In this case, Leu and Phe was chosen, as the P1 site of calpain is known to accommodate bulky amino acids, as follows:

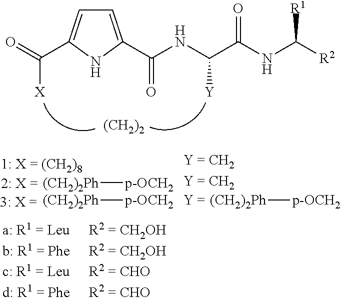

Macrocyclic peptidomimetic template used in this study. Leu=leucine, Phe=phenylalanine.

The efficacy of the inhibitors was evaluated using ovine calpains in in vitro assays. The ovine model was chosen over the rodent models as the ovine lens proteins were found to be more similar to human lens protein then those of rats, as shown by the close homology between sequences of ovine and human crystalline. Our inhibitors were tested against ovine calpain 2, with the most potent tested against ovine calpain 1 to determine if selectivity existed between the two calpain isoforms. In addition, inhibitors were tested against bovine β-chymotrypsin (serine protease), which is known to accommodate a Phe group in the P1 site, allowing evaluation of selectivity of our inhibitors across classes of proteases and to check the versatility of our inhibitor design.

Synthesis of macrocycles 1-3 was based on RCM of dienes 13-15, which were prepared from readily available, simple building blocks (Scheme 2). The synthesis of the required dienes was divided into the preparation of two building blocks, 1. modified hydroxylphenyl 7 and 2. tethered pyrrole 10 (Scheme 1). Initially, 7 was prepared by reaction of 4 with allyl bromide to give 5, which was hydrolyzed to 6 and treated with thionyl chloride to give the required acid chloride 7. 10 was prepared from acetylation of pyrrole, 8 with trichloroacetyl chloride to give 9, which was treated with sodium ethoxide to give the required ester 10. Ester 10 was then reacted separately with 10-undecenoyl chloride and acid chloride 7 in the presence of Yb(OTf)₃ to give 11a and 12a, which were hydrolyzed to the required acids 11b and 12b, respectively in moderate yields. The required diene 13 was prepared by coupling acid 11b with (S)-allyl-Gly-OMe(*), while dienes 14 and 15 were similarly prepared from acid 12b, by treatement with (S)-allyl-Gly-OMe(*) and (S)-allyl-Tyr-OMe(**), respectively (Scheme 2).

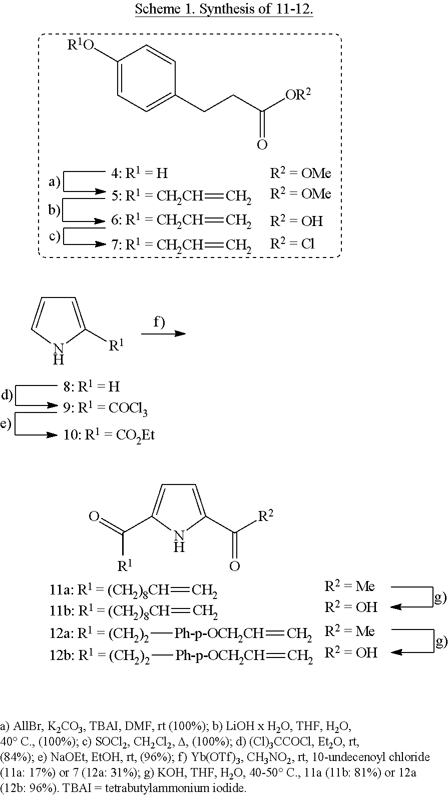

With the requisite dienes in hand, RCM of 13-15 was performed using Grubbs second-generation catalyst to give the required macrocycles in moderate yields. RCM of acyclic dienes 13-16 gave mixtures of cis/trans alkenes, which were hydrogenated directly to macrocyclic esters 17-19. Macrocyclic esters 17-19 were subsequently hydrolysed to their corresponding carboxylic acids, followed by peptide coupling with either (L)-leucinol or (L)-phenalaninol to give macrocyclic alcohols 1a-3a and 1b-3b, respectively. Alcohols 1a-3a and 1 b-3b were then oxidized with Dess-Martin reagent to give aldehydes 1c-3c and 1 d-3d in moderate yields (Scheme 2).

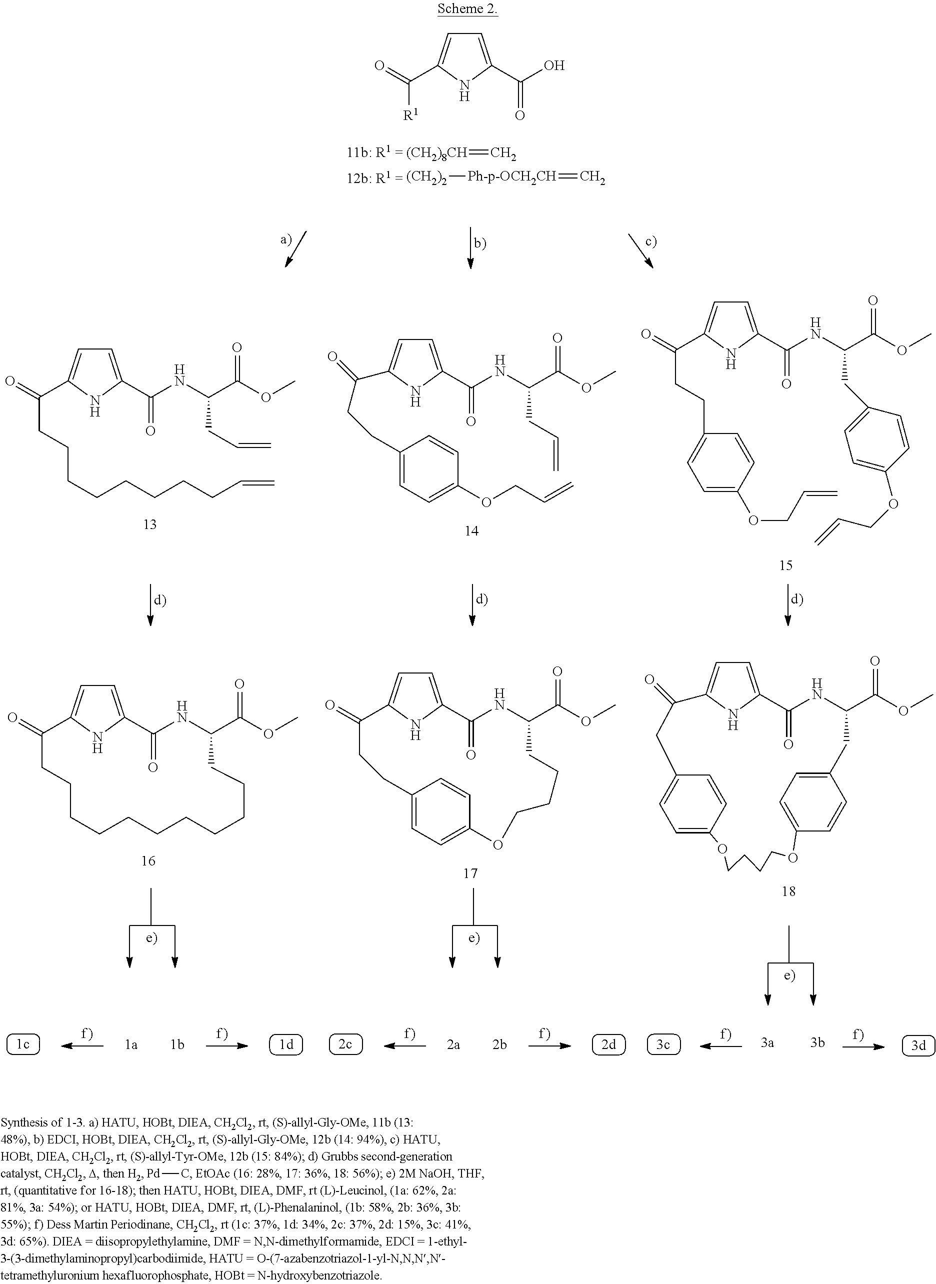

The alcohols and aldehydes, 1-3, were assayed against ovine calpain 2 (o-CAPN2) and bovine α-chymotrypsin (bCT) to determine in vitro potency and protease selectivity. Ovine calpain 1 (o-CAPN1) assays were also performed for the most potent compounds to determine if isoform selectivity existed. All alcohols 1a-3a and 1b-3b were found to be inactive against o-CAPN2 and bCT (data not shown) indicating the absence of non-covalent inhibition and that the presence of an aldehyde group is preferred for covalent inhibition of the proteases. The most potent o-CAPN2 inhibitor was found to be the 18-membered macrocyclic aldehyde, 1c, which is presumably due to the greater flexibility of the ring and the presence of a Leu group in P1 (IC50=66 nM). All other macrocyclic compounds were found to be equally potent against o-CAPN2, ranging from 150-250 nM. This suggests that the incorporation of a pyrrole ring into the peptide backbone allowed the adoption of the preferred β-strand conformation for binding.

Table 1 shows the in vitro inhibition assay data for ovine calpain and bovine α-chymotrypsin.

TABLE 1

| | o-CAPN1 | o-CAPN2 | bCT |
|---|---|---|---|
| Compound | (IC⁵⁰ [nm])^{[a]} | (IC⁵⁰ [nm])^{[a]} | (Kᵢ [nm])^{[b], [c]} |
| 1c | 42 ± 5 | 66 ± 8 | NI^{[e]} |
| 2c | 324 ± 80 | 249 ± 50 | NI^{[e]} |
| 3c | ND^{[d]} | 203 ± 40 | 1917 ± 167 |
| 1d | ND^{[d]} | 156 ± 27 | 2525 ± 248 |
| 2d | ND^{[d]} | 153 ± 20 | 431 ± 5 |
| 3d | ND^{[d]} | 246 ± 60 | 33 ± 5 |

In Table 1: ^{[a]} Values are the mean of two experiments in triplicate. Variation between experiments is less than ±10% ^{[b]} Values are the mean of three experiments. Variation between experiments is less than ±10% [c] Inhibition studies we performed at 0.3×Km and 0.75×Km ^{[d]} Not determined ^{[e]} No inhibition, highest inhibitor concentration analysed (25-125 μM). This correspond to the maximum amount of compound soluble under these conditions.

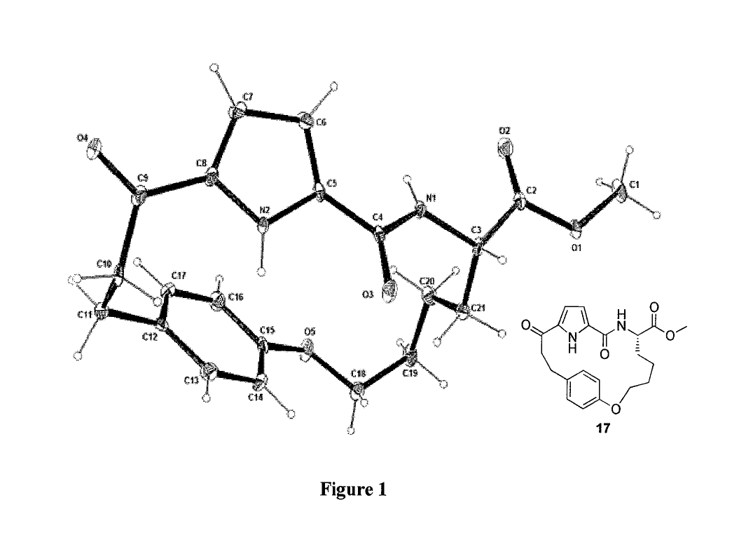

By comparing the two 18-membered macrocycles 1c and 2c, the decrease in potency of 2c was most likely due to the rigidity of the compound with the introduction of a phenyl group into the ring. X-ray crystallography of 17 supported this suggestion showing the rigidity of the 18-membered ring system (FIG. 1). Interestingly, compound 3c was found to be just as potent as 2c, even with its large ring system (24-membered). The introduction of two phenyl rings into the ring system of 3c may have increased its rigidity and despite its large size, was able to fit into the binding pocket of calpain in the desired orientation.

Comparison of 1c and 1d, which only differs in the P1 group, it can be seen that Leu is preferred to Phe at the P1 position. In addition, protease selectivity between o-CAPN2 and bCT was observed upon changing the P1 group from Phe to Leu. While all macrocyclic aldehydes were found to be active against o-CAPN2, all compounds that have Phe in P1 were also found to be potent against bCT. Hence, protease selectivity can be ensured by having Leu in P1. Potent o-CAPN2 inhibitor, 1c and for comparison, the 18-membered macrocyclic inhibitor, 2c were assayed against o-CAPN1. The assay results showed no isoform selectivity for inhibitor 1c (IC50=66 nM (o-CAPN2) vs. 42 nM (o-CAPN1)) or inhibitor 2c (IC50=249 nM (o-CAPN2) vs. 324 nM (o-CAPN1)).

In conclusion, the synthesis and biological evaluation of 18- and 24-membered macrocycles has identified potent inhibitors for calpain. The incorporation of a pyrrole moiety into the backbone design ensured a β-strand conformation, and a decrease in peptide-like nature of the inhibitors. Aldehyde 1c proved to be the most potent calpain inhibitor, with a more flexible ring system. Through modification of the P1 group, selectivity of the inhibitors can be designed for different protease groups.

### Example 4

### New 26S-Proteasome Inhibitors with High Selectivity for Chymotrypsin-Like Activity and p53-Dependent Cytotoxicity

Here, we report the synthesis and characterization of new tripeptide aldehydes that are designed for increased specificity for chymotrypsin-like (CT-L) activity of the proteasome. In contrast to the benchmarks, MG132 and Bortezomib, these analogues are highly specific inhibitors of CT-L activity and show potent activity in sarcoma cell lines without non-specific cytotoxicity to normal non-malignant cells. Our findings suggest that the p53 pathway is a major effector in the ability of these novel proteasome inhibitors to induce cell death.

Results and Discussion

Inhibitor Design

Although there are numerous CT-L specific inhibitors reported few of these have been extensively studied and tested as anti-cancer agents in vivo. Encouragingly, 2-aminobenzyl statine-based inhibitors, which display selectivity and reasonable potency for CT-L activity, show high antiproliferative activity in cell-based assays. It has been reported that the introduction of a sterically bulky substituent at P2 of peptidic aldehydes enhances selectivity for CT-L activity, where the corresponding S2 pocket is ill-defined and thought not critical for binding. One such example, with a Asp(t-Bu) at P2, shows modest potency and selectivity for CT-L over T-L, CP-L and also a cysteine protease calpain. Further improvements are required to better tailor the activity of peptidic aldehydes towards the CT-L subunit of the proteasome, while also reducing their activity against off-target proteases.

The inhibitors reported here (Compounds 1-6, see below) have been developed based on the structure of the bench-mark peptidic aldehyde inhibitor MG132, which shows high potency against all three proteasome activities (see Table 1).

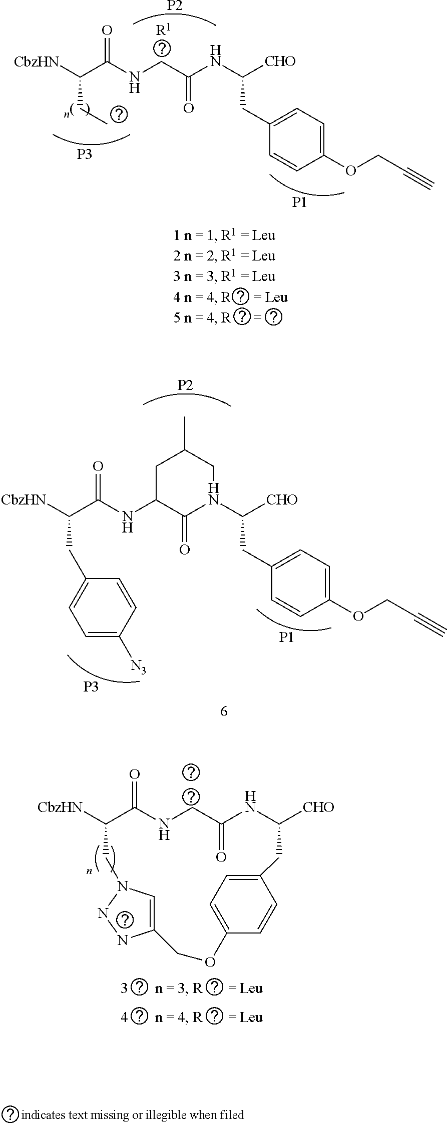

### Tripeptide MG132 Analogues

We chose to incorporate an acetylene substituted aryl group at P1, as the corresponding S1 pocket of the CT-L subunit is known to bind hydrophobic groups. By comparison, the importance of P3 to binding is less well studied, with the make-up of the corresponding S3 binding pocket known to vary between the three different activities. We saw this site as a relatively unexplored opportunity to introduce selectivity into the inhibitors. Here, we report the incorporation of an aliphatic azide (as in 1-5) or an aromatic azide (as in 6) at P3 as previously untested substituents at this position. These azides also allow cyclisation to the acetylene of P1 via Huisgen cycloaddition, in order to investigate the effect of constraining the backbone into a β-strand geometry (see compounds 3a and 4a). This geometry is likely to favour ligand binding to the proteasome and indeed all other proteases. While inhibitors of the proteasome adopt hydrogen bonds with the protease that are characteristic of binding in this geometry, unlike other proteases, the P2 group does not seem to form important contacts with the active site. Presumably this accounts for the earlier observations that the corresponding S2 pocket is not critical for binding to CT-L subunit. We chose to incorporate both Leu and a subtle variant (Ile) at P2 of our new inhibitors to allow direct comparison with MG132.

### Synthesis of Tripeptide MG132 Analogues

The tripeptide aldehydes 1-6 were prepared by standard peptide coupling as depicted in Schemes 1 and 2. Separate reactions of 7-10 with Leu-OtBu or IIe-OtBu, in the presence of EDCI and HOBt, gave dipeptides 12-16, the tert-butyl esters of which were hydrolyzed to give the carboxylic acids 17-21. Coupling of each of these with the amino alcohol 11, in the presence of EDCI and HOBt, gave tripeptides 22-26 that were oxidised with Dess-Martin periodinane (DMP) to give the required acyclic aldehydes 1-5 respectively. The tripeptides 24 and 25 were also cyclized on treatment with Cu(I)Br in CH2Cl2 to give 27 and 28, which were oxidized with DMP to give 3a and 4a. An analogous reaction of the derivatives with a shorter tether (22 and 23) failed to give corresponding macrocycles, presumably because of steric strain associated with the corresponding smaller ring systems. The macrocycles of 3a and 4a had been shown in earlier work to con-strain the geometry of the peptide backbone into the required β-strand geometry.

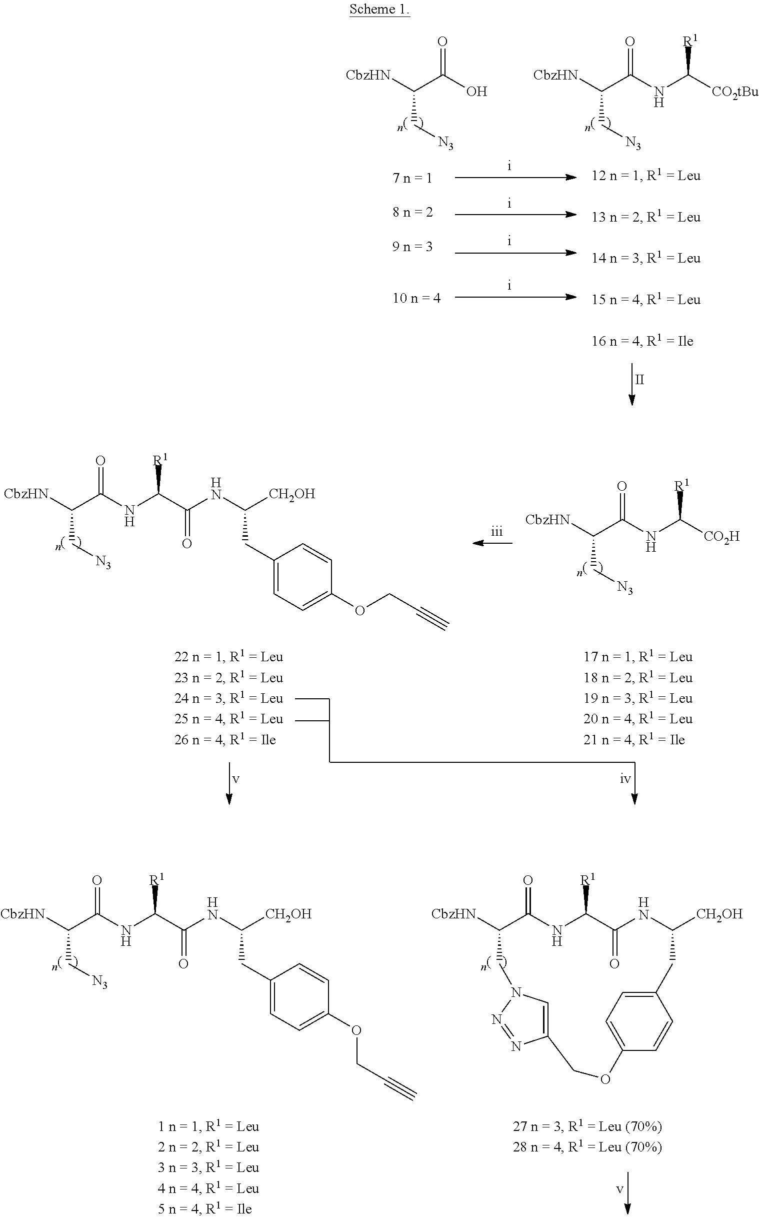

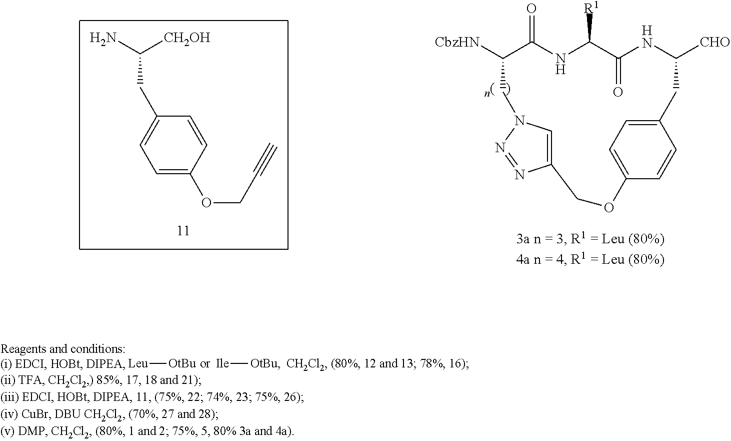

Compound 6, with an aryl group at both P1 and P3 was similarly prepared as shown in Schemes 2. Reaction of 29 with Leu-OtBu, in the presence of EDCI and HOBt, gave dipeptide 30 that was hydrolyzed to give 31. Coupling of this carboxylic acid with the amino alcohol 11, in the presence of EDCI and HOBt, gave tripeptide 32, the aldehyde of which was oxidised with DMP to give the required aldehyde 6. The attempted cyclization of 32, on treatment with Cu(I)Br in CH₂Cl₂, failed to give the corresponding macrocycle, again presumably due to steric constraints of the associated ring.

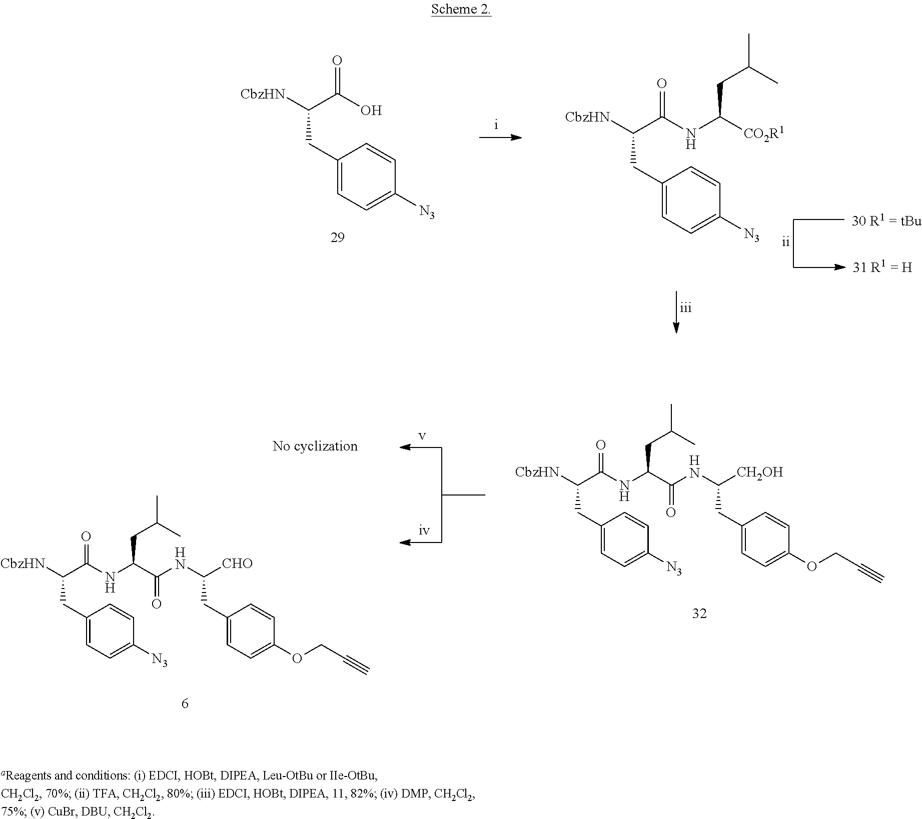

Inhibition of the Proteasome

We initially determined the ability of the novel tripeptide MG132 analogues 1-6 and the macrocycles 3a and 4a to inhibit the three separate protease activities of the 20S proteasome (CT-L, -T-L and CP-L). The assays were performed in vitro using purified 20S proteasome/inhibitor mixtures, with the activity of each subunit of the proteasome assessed upon incubation with its respective target peptide. MG132 and Bortezomib were highly potent inhibitors of CT-L activity in this assay, see Table 1. However, MG132 also significantly inhibited the T-L and CP-L activities of the 20S proteasome, which is consistent with previous reports. The new peptidic aldehydes 1-6 were also highly potent against the CT-L activity, with derivatives 5 and 6 proving to be the most potent inhibitors (IC₅₀ values of 21 nM and 23 nM, respectively). However, unlike Bortezomib and MG132, all the compounds (1-6) were inactive against both the T-L and CP-L activities of the proteasome up to the highest concentrations tested (25,000 nM). It is interesting to note that the introduction of Ile at P2 (see compound 5) gave rise to a 7.5 fold increase in potency against the CT-L proteolytic activities relative to the direct MG132 analogue with Leu at P₂ (see compound 4). In addition, there is no apparent clear-cut preference for the length of tether (compare compounds 1 to 4), while the introduction of an aryl group at P3 (as in 6) is well tolerated.

The cyclised derivatives (3a and 4a) were on average over 10 fold less potent against CT-L than their acyclic counterparts 3 and 4 (Table 1). Thus, it appears that the proteasome prefers to bind a conformationally flexible acyclic ligand, rather than a structure that has its peptide backbone constrained into a β-strand conformation. This contrasts other proteases such as calpains, where cyclization into a β-strand significantly increases potency (Table 1). Thus, cyclization of peptide aldehydes provides a novel avenue to dictate specificity between the proteasome and calpain proteases.

The combination of high potency and selectivity for the CT-L activity of the proteasome observed for the tripeptidic aldehydes compounds 1, 3, 5 and 6 is rarely observed. With the exception of a series of tripeptide-based vinyl sulfones and some α-keto amides, most inhibitors that show some selectivity for CT-L lack potency. Here, we show for the first time that the combination of high potency and selectivity for CT-L can be achieved with appropriate modification at P1 and P3 of MG132. Carfilzomib, a proteasome inhibitor in phase IIb trials, is the only proteasome in current clinical testing that has been experimentally shown to elicit specificity for the CT-L subunit. However, such CT-L specificity was only observed at low doses of calfilzomib, as higher doses inhibited all three activities of the 20S proteasome. Interestingly this peptide-based inhibitor has a C-terminal epoxide, aryl groups at P2 and P4, and Leu at P1 and P3.

TABLE 1
Proteasome Inhibitory and Calpain II Activity (standard
errors are all less than 5% of the mean)

| | | n | | IC₅₀ (nM) | | | |
|---|---|---|---|---|---|---|---|
| Compound | P2 | (P3) | Cyclic | CT-L | T-L | CP-L | Calpain II |
| 1 | Leu | 1 | N | 34 | >25,000 | >25,000 | 107 |
| 2 | Leu | 2 | N | 355 | >25,000 | >25,000 | 324 |
| 3 | Leu | 3 | N | 54 | >25,000 | >25,000 | 780 ^{b} |
| 4 | Leu | 4 | N | 150 | >25,000 | >25,000 | 1,030 ^{b} |
| 5 | IIe | 4 | N | 21 | >25,000 | >25,000 | 389 |
| 6 | Leu | ^{a} | N | 23 | >25,000 | >25,000 | nd^{d} |
| 3a | Leu | 3 | Y | 917 | >25,000 | >25,000 | 137^{e} |
| 4a | Leu | 4 | Y | 250 | >25,000 | >25,000 | 97^{e} |
| MG132 | Leu | ^{b} | N | 1.2 | 1998 | 545 | 311 |
| Bortezomib | Phe | ^{c} | N | 35 | >25,000^{f} | 438 | >25,000 |

^{a} azido-Phe at P3.
^{b} Leu at P3.
^{c} no P3 amino acid.
^{d}nd = Not determined.
^{e}Previously reported data.
^{f}Bortezomib activated T-L activity by 32%.

Tripeptide MG132 Analogues Specifically Kill Cancer Cells

We next investigated whether the combination of high potency and selectivity for CT-L, possessed by our inhibitors, translated into improved cytotoxic activity against cultured cancer cell lines. The viability of a panel of four sarcoma cell lines was determined following 48 hours exposure to a titration of concentrations of Bortezomib, MG132, acyclic compounds 1 to 6 or cyclic compounds 3a and 4a (Table 2). Parallel viability studies were performed using either normal primary human fibroblasts or primary osteoblasts, thus allowing us to determine if the cytotoxicity of these compounds was cancer cell-specific and hence determine an in vitro therapeutic window for each compound (Table 2).

Compounds 3, 5 and 6 were highly potent against cancer cells, with average 10₅₀ values (1.6, 0.88 and 1.5 μM, respectively) comparable to that of MG132 (0.61 μM). In fact, these three compounds showed higher levels of cytotoxicity than their precursor MG132 in some cancer cell lines. Importantly and in contrast to MG132, the cytotoxicity of these compounds was more specific to cancer cells, with a significantly reduced toxicity to the normal cell lines. In particular, compound 6 showed 19 fold more potency against cancer cells over normal cells, effectively increasing the therapeutic window of MG132 by over 4 fold. In fact, both compounds 5 and 6 showed an increased specificity for cancer cell lines over normal cells than the two benchmark proteasome inhibitors, MG132 and Bortezomib. The macrocyclic compounds 3a and 4a were all significantly less potent against cancer cells compared to their acyclic counterparts. This is expected given their lower CT-L in vitro activity as discussed earlier.

The importance of specifically targeting the CT-L subunit as an anti-cancer therapy is still unresolved, with conflicting reports in the literature. For example, our finding of anti-neoplastic activity with CT-L specific inhibitors is consistent with a previous report from Parlati et al. that demonstrated that specific inhibition of the CT-L subunit using carfilzomib drove selective cell death in multiple myeloma, non-Hodgkin's lymphoma and leukemia cell lines. However, it's important to note that carfilzomib also inhibited LMP7, the similar subunit corresponding to the CT-L site in the immunoproteasome of these cells. In contrast, a comprehensive study from the Kisselev laboratory demonstrated that co-inhibition of CT-Lsubunit with either the T-L or CP-L subunits was necessary to achieve maximal cytotoxic activity of multiplemyeloma cell lines. These observations are not consistent with our findings herein using sarcoma cell lines, thus potentially underscoring the fundamental differences in the proteasome function between solid tumours (sarcomas) and hematological malignancies (multiple myeloma). Furthermore, the mechanisms of action of proteasomal inhibitors may be disease-specific; in particular in relation to malignancies such as multiple myeloma that are associated with an excessive amount of mis-folded proteins and hence are more heavily reliant recycler function of the proteasome.

Thus the acyclic derivatives reported here have a superior therapeutic window compared to the benchmark inhibitors, MG132 or Bortezomib. Importantly, several of these new compounds combine a high potency for the CT-L activity of the proteasome with an improved ability to selectively kill cancer cells compared to MG132.

TABLE 2
Cyto-toxicity of proteasome inhibitors against a panel of sarcoma cell lines
or normal cell lines (standard errors are all less than 5% of the mean)

| | | | | | | | | Average | |
|---|---|---|---|---|---|---|---|---|---|
| | | | | | Average | | | IC₅₀ for | |
| | Cancer cell lines | | | | IC₅₀ for | Normal cell lines | | normal | |
| | IC₅₀ (μM)^{a} | | | | cancer cell | IC₅₀ (μM)^{a} | | cell lines | Therapeutic |
| Compound | WE-68 | VH-64 | STA-ET-1 | TC-252 | lines (μM) | Fibroblasts | Osteoblasts | (μM) | window^{b} |
| 1 | 4.5 | 5.8 | 6.5 | 5.0 | 5.4 | 12.2 | 23.7 | 18.0 | ×3.3 |
| 2 | 4.7 | 4.9 | 5.4 | 2.3 | 4.3 | 29.4 | 13.6 | 21.5 | ×5.0 |
| 3 | 0.47 | 2.1 | 2.6 | 1.2 | 1.6 | 8.8 | 18.8 | 13.8 | ×8.7 |
| 4 | 10.8 | 1.9 | 2.6 | 1.6 | 4.2 | 39.5 | 10.4 | 25.0 | ×5.9 |
| 5 | 0.98 | 1.1 | 1.0 | 0.42 | 0.88 | 19.9 | 4.8 | 12.4 | ×14.1 |
| 6 | 1.1 | 1.9 | 2.4 | 0.64 | 1.5 | 45.4 | 11.6 | 28.5 | ×19.0 |
| 3a | 30.5 | 12.8 | 6.2 | 5.4 | 13.7 | >50 | >50 | — | — |
| 4a | 18.0 | >50 | 37.6 | 39.4 | 31.7 | >50 | >50 | — | — |
| MG132 | 0.68 | 0.68 | 0.59 | 0.49 | 0.61 | 2.8 | 2.7 | 2.8 | ×4.5 |
| Bortezomib | 0.02 | 0.04 | <0.0008 | 0.01 | 0.02 | 0.02 | 0.28 | 0.15 | ×9.1 |

^{a}Dose-response curves not shown.
^{b}The ‘Therapeutic window’ represents the fold change in potency (IC₅₀ value) of the proteasome inhibitor against the cancer cell line versus the normal cell line.

Tripeptide MG132 Analogues Mediate Cell Death in Part Through the p53 Pathway.

Although proteasome inhibitors have been translated from the bench to the clinic over the past decade, their specific mechanisms of action remain poorly understood. As discussed earlier, proteasomal over-activity in tumor cells has been attributed to aberrant degradation of numerous critical cancer-related pro proteins, including the pro-apoptotic tumor suppressor, p53. However, the role of p53 as a downstream mediator of cell death in response to proteasomal inhibition remains unclear. Therefore, we used our small library of CT-L specific proteasome inhibitors (compounds 1-6) to determine the role of p53 in their mechanism of cytotoxic action. For this purpose, we utilized mouse embryonic fibroblasts (MEFs) from transgenic p53^{+/+} and p53^{-/-} littermates. This isogenic pair of cell lines are either competent (MEF p53^{+/+}) or deficient (MEF p53^{-/-}) in p53 protein and are used here to assess a potential role for p53 as a downstream inducer of cell death in response to proteasomal inhibition.

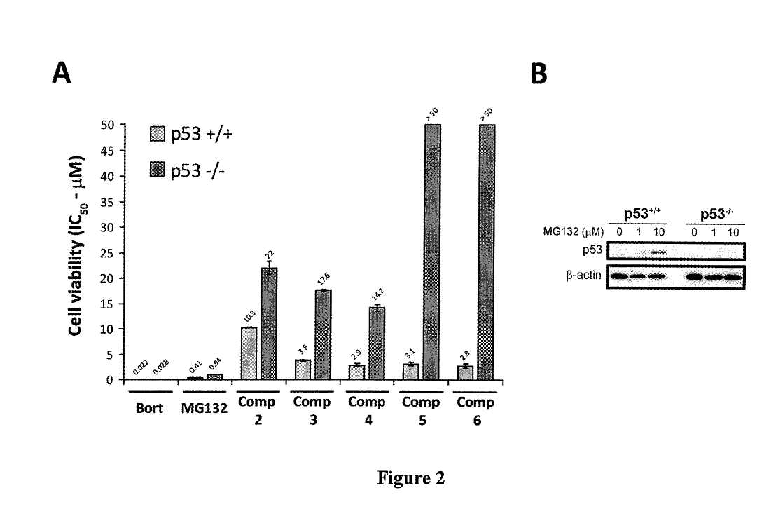

The p53^{+/+} MEFs were moderately sensitive to compounds 2, 3, 4, 5 and 6, with IC₅₀ values ranging from 2.7 to 10.3 μM (FIG. 2). In contrast, MEFs lacking p53 (p53^{-/-}) were significantly less sensitive to the cytotoxic effects of these compounds, with deficiency of p53 rendering MEFs resistant to the cytotoxic activities of compounds 5 and 6. A similar trend for p53-dependent cytotoxicity was observed upon treatment with MG132. MEF p53^{+/+} cells were over 2-fold more sensitive to MG132 (IC₅₀ value of 0.41 μM) as compared to the MEF p53^{-/-} counterparts (IC₅₀ value of 0.94 μM). Importantly, exposure of MEFs p53^{+/+} to MG132 was also associated with increase p53 protein levels (FIG. 3B), demonstrating that proteasomal inhibition results in accumulation of p53 protein in these cells. Collectively, the data demonstrate that inhibition of the proteasome using tripeptide aldehydes is associated with stabilization of biologically active p53, leading to cell death.

These studies on an isogenic pair of p53^{+/+} or p53^{-/-} cells demonstrate the contribution of p53 as a critical mediator of cell death following proteasomal inhibition. The role of p53 in this process was previously unclear, with reports suggesting that p53 is either essential or dispensable for the anti cancer activity associated with proteasomal inhibition. Interestingly, the reports that refute a role for p53 as a cytotoxic mediator following proteasomal inhibition are restricted to hematological malignancies and generally involve the use panel cell lines with diverse genetic backgrounds that may compromise any p53-related response, rather than an isogenic system presented herein. Our conclusions of a p53-dependent mode of cytotoxic action is further strengthened by consistent results across a small library of proteasome inhibitors (Bortezomib, MG132 and compounds 2 to 6) and are not limited to the use of a single proteasome inhibitor.

In summary, we have shown that the incorporation of an azide group at P3 in acyclic aldehydes results in compounds (see 1 to 6) with a high degree of selectivity for CT-L over both T-L, CP-L. Further work is required to define the exact role of the azide group, but the effect is significantly more pronounced than that observed for the literature peptidic aldehyde MG132. Of general significance to future inhibitor design is the observation that the incorporation of Ile, in place of Leu, at P2 significantly increases potency against the CT-L proteolytic activity. Huisgen cycloaddition of the P3 azide with an aryl acetylene at P1 gave rise to macrocycles constrained into a β-strand geometry. Unlike reports for other proteases (particularly calpain II as discussed here), this gave compounds with reduced potency and also reduced anti-cancer efficacy. This is a unique and significant finding that may reflect an earlier observation that the S2 pocket is not critical for binding to CT-L subunit. Thus, specific cyclization of peptide aldehydes provides a novel avenue to dictate specificity between the proteasome and other proteases.

The new acyclic CT-L specific inhibitors (compounds 1 to 6) were significantly more specific for cancer cells compared to MG132. In particular, the addition of the aromatic azide at P3 (compound 6) was associated with a 10 fold improvement of the therapeutic window of MG132. Lastly, we have used this new panel of potent proteasome inhibitors to demonstrate a critical role of the p53 tumor suppressor protein as a mediator of the cytotoxic response associated with proteasomal inhibition.

Materials and Methods

Chemicals: MG132 (Sigma-Aldrich, St Louis, Mo., USA), Bortezomib (LKT Laboratories, St Paul, Minn., USA) or MG132 derivatives were dissolved in 10 mM DMSO and stored at −20° C.

In Vitro Proteasome Activity Assay

Purified rabbit 20S proteasome and fluorogenic CT-L substrate (Suc-LLVY-AMC) were purchased from Boston Biochem (Cambridge, Mass., USA). The T-L and CP-L fluorogenic substrates (Ac-RLR-AMC and Z-nLPnLD-AMC) were purchased from Enzo Life Sciences (Farmingdale, N.Y., USA). The 20S proteasome was diluted to 0.2 μg/μL in 20S proteasome buffer (50 mM HEPES pH 7.6, 150 mM NaCl and 1 mM DDT) and stored at −80° C. Purified 20S proteasome (8 ng) was pre-incubated with the indicated concentrations of inhibitors for 15 minutes and subsequently added to the AMC-labeled substrate peptide (50 μM) in assay buffer (25 mM HEPES, pH 7.5, 0.5 mM EDTA, 0.05% NP-40, and 0.001% SDS (w/v)) at 37° C. for 2 hours. Fluorescent substrate cleavage by the 20S proteasome was linear during this incubation timeframe (Supplementary Figure S4. Hydrolysed 7-amino-4-methylcoumarin (AMC) was subsequently detected with the FLUOstar OPTIMA microplatefluorometer at excitation/emission of 390/460 nm. The activity was estimated in relative fluorescence units and half of the maximal inhibitory activity of the proteasome is represented by IC₅₀ values. A minimum of three biological replicates were performed for each data point.

Cell Viability Assays

Cell viability assays were performed as previously described. Briefly, cells were seeded in 96 well microtiterplates at a density of 3×10⁴ cells per well in the presence of the indicated chemical. Cells were harvested 48 hours post-treatment, centrifuged at 1,300×g, washed in phosphate-buffered saline (PBS) and stained with 7AAD solution (2 μg/mL) (7-amino-actinomycin-D, Invitrogen, Carlsbad, Calif.) for 10 mins at room temperature. Viable cells were determined with the use of a FACS Calibur flow cytometer (Becton Dickinson Immunocytometry Systems), and analyzed with the use of FLOWJO (Tree Star, Inc) and GraphPad Prism (GraphPad Software Inc).

Cell Lines and Culture Conditions

WE-68 and VH-64 Ewing's sarcoma cells were kindly supplied by F. van Valen (Department of Orthopaedic Surgery, Westfalische-Wilhelms-University, Germany).TC-252 and STA-ET-1 Ewing's sarcoma cells were kindly provided by G. Hamilton (Department of Surgery, University of Vienna, Austria) or P. Ambros (Children's Cancer Research Institute, St Anna Children's Hospital, Vienna, Austria). Primary human embryonic fibroblasts or primary human osteoblasts were collected from the Women's and Children's Hospital (North Adelaide, South Australia, Australia) or Royal Adelaide Hospital with patient consent. Mouse Embryonic Fibroblasts (MEFs) and its p53-null derivative were kindly supplied by Guillermina Lozano (Department of Genetics, Anderson Cancer Centre, University of Texas, Houston, Tex., USA). WE-68 cell lines were grown in RPMI-1640 media, whilst human embryonic fibroblasts and MEFs were grown in Dulbecco's Modified Eagle's Medium (DMEM). Media was supplemented with 10% FCS, 1% PSG and 10 mM HEPES. All cells were maintained at 37° C. in a humidified atmosphere of 5% CO₂.

Western Blotting

Western blot analyses were performed as previously described. Briefly, cells were harvested and lysed in 50 mM Tris-HCl (pH 7.5), 250 mM NaCl, 1 mM EDTA, 50 mM NaF, 0.5% TritonX-10, 0.5 mM Na₃VO₄ and 1× proteasome inhibitor (Roche, Indianapolis, Ind., USA). Lysates were incubated on ice for 8 minutes and sonicated using a Vibra-Cell VCX130 (Sonics & Materials, Inc) at 25% amplitude for 10 seconds. Protein concentrations were assayed with the bicinchoninic acid assay (Thermo Scientific, Massachusetts, USA) and subsequently resolved using a 10% SDS-PAGE gel. Proteins were transferred onto a nitrocellulose membrane (Hybond-C Extra, Amersham Biosciences), blocked (10% milk/TBST; 30 mins), hybridized with the appropriate primary or HRP-conjugated secondary antibody and subsequently visualized using Enhanced Chemiluminescence (Amersham Biosciences).

Antibodies

Antibodies used included a mouse anti-β-actin (Sigma), anti-p53 (1C12-mouse specific) (Cell Signalling, Danvers, Mass., USA), sheep anti-mouse IgG-HRP (Amersham Biosciences, Piscataway, N.J., USA), or donkey anti-rabbit IgG-HRP (Amersham Biosciences).

### Example 5

### In Vitro Lens Culture Assay

The ability of the macrocyclic compounds as described to prevent the formation of a calcium induced cataract in adult ovine lens may be assayed using the procedure of J. Sanderson, J. M. Marciantonio and G. A. Duncan (2000) Invest. Opth. Vis. Sci. 41: 2255.

For example, a number of pairs of lenses may be tested. One lens from each pair may be preincubated with 1 μM of a compound in EMEM-culture media, for 3 h while the other may be incubated at 35° C., 5% CO2. Then 5 mM calcium chloride may be added onto both the inhibitor treated lens and the other lens, and both lenses then incubated for 20 h. The lenses may be photographed and the images digitally analysed for opacity.

### Example 6

### In Vivo Tests

An ointment (50 mg) comprising 1% of a macrocyclic compound as described herein may be applied to one eye of a lamb, three times in one day, and any signs of irritation monitored.

Lambs genetically predisposed to cataracts may be used. An ointment (25 mg) comprising 1% of a macrocyclic compound as described herein may be applied twice daily to the right eye of one group of lambs for three months starting when they are three to four months old. A placebo ointment (25 mg) may also be applied twice daily to the right eye of another group of lambs for three months starting when they are three to four months old.

The progression of cataracts may be determined by a veterinary ophthalmologist with a slit-lamp microscope.

### Example 7

### Formulations

Ointment

An ointment, suitable for intraocular application, and having the following composition (w/w) may be prepared:

1% compound of a macrocyclic compound as described herein

25% cetyl stearyl alcohol

35% wool fat

39% paraffinum subl.

Cetyl stearyl alcohol may be heated until it has melted. The compound may then be added and the oil stirred until the compound dissolved. Wool fat and paraffinum subl. May then be added and the mixture heated until all the components have melted. The mixture is allowed to cool with constant stirring until an ointment forms.

Emulsion

An emulsion, suitable for intraocular application, and having the following composition (w/w) may be prepared according to the procedure described below:

0.7% compound of a macrocyclic compound as described herein

20% cetyl stearyl alcohol

25% wool fat

25% paraffinum subl.

1% sodium lauryl sulfate

0.1% sodium benzoate

28.3% water

The hydrophobic phase (cetyl stearyl alcohol, wool fat, paraffinum subl.) and the hydrophilic phase (sodium lauryl sulfate, sodium benzoate, water) may be separately heated to 50° C. The compound may then be added to the hydrophobic phase and stirred until the compound is dissolved. The hydrophilic phase may then be added to the hydrophobic phase, and the heating source removed. The mixture may then be stirred until it reaches room temperature. The resulting emulsion is then checked for the absence of crystals by differential scanning calorimetry at the melting point of the compound used.

### INDUSTRIAL APPLICATION

It will be appreciated from description herein that the present disclosure provides novel compounds having protease inhibitory properties. These compounds may be formulated into medicament for use in a therapeutic application for which their activity makes them appropriate, such as in the prevention and/or treatment of cataracts. The compounds as described herein also have application as inhibitors of proteases for research and/or testing purposes.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Although the present disclosure has been described with reference to particular examples and embodiments, it will be appreciated by those skilled in the art that the disclosure may be embodied in many other forms.

1. A compound of Formula I and/or a pharmaceutically acceptable salt, solvate, tautomer, hydrate or prodrug derivative thereof: 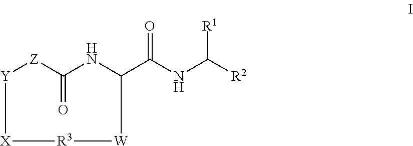
wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

2.-23. (canceled)

24. The compound according to claim 1, wherein the compound has one of the following formulas and/or a pharmaceutically acceptable salt, solvate, tautomer, hydrate or prodrug derivative thereof:

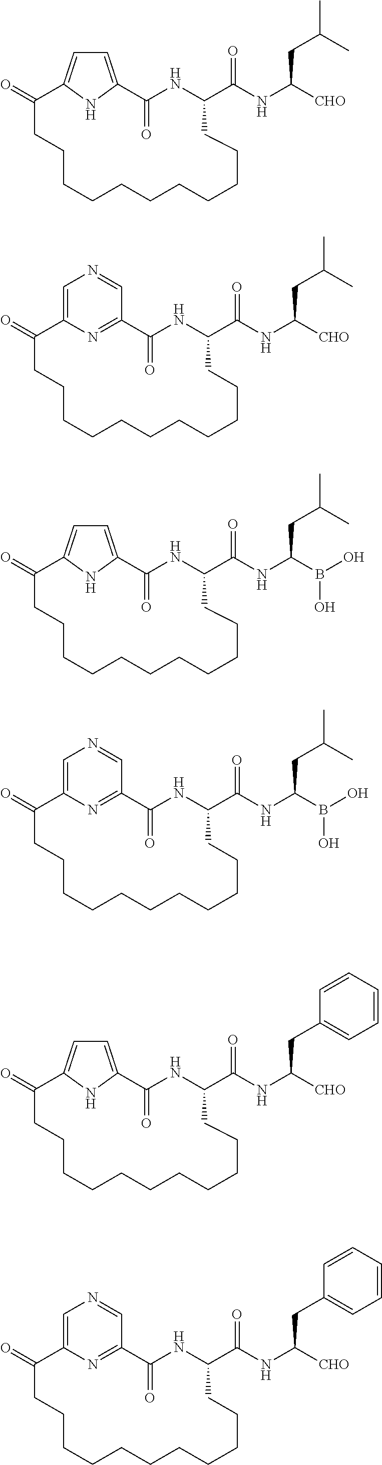 [SMILES: CC(C)C[C@@H](C=O)NC(=O)[C@@H]1CCCCCCCCCCCC(=O)c2ccc([nH]2)C(=O)N1 ; CC(C)C[C@@H](C=O)NC(=O)[C@@H]1CCCCCCCCCCCC(=O)c2cncc(n2)C(=O)N1 ; CC(C)C[C@H](NC(=O)[C@@H]1CCCCCCCCCCCC(=O)c2ccc([nH]2)C(=O)N1)B(O)O ; CC(C)C[C@H](NC(=O)[C@@H]1CCCCCCCCCCCC(=O)c2cncc(n2)C(=O)N1)B(O)O ; O=C[C@H](Cc1ccccc1)NC(=O)[C@@H]1CCCCCCCCCCCC(=O)c2ccc([nH]2)C(=O)N1 ; O=C[C@H](Cc1ccccc1)NC(=O)[C@@H]1CCCCCCCCCCCC(=O)c2cncc(n2)C(=O)N1]

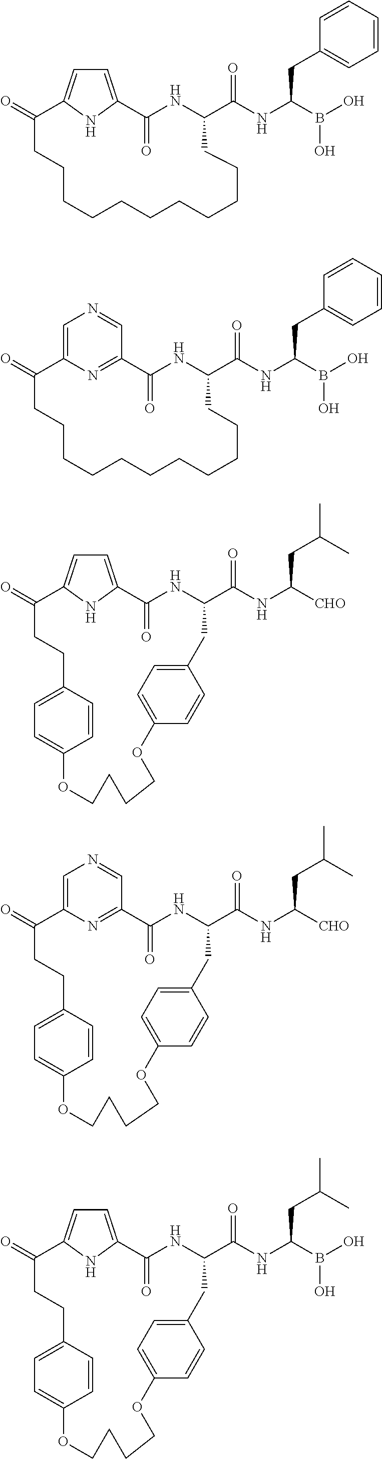 [SMILES: O=C1CCCCCCCCCCC[C@@H](C(=O)N[C@@H](Cc2ccccc2)B(O)O)NC(=O)c2ccc1[nH]2 ; O=C1CCCCCCCCCCC[C@@H](C(=O)N[C@@H](Cc2ccccc2)B(O)O)NC(=O)c2cncc1n2 ; CC(C)C[C@@H](C=O)NC(=O)[C@@H]1Cc2ccc(cc2)OCCCCOc2ccc(cc2)CCC(=O)c2ccc([nH]2)C(=O)N1 ; CC(C)C[C@@H](C=O)NC(=O)[C@@H]1Cc2ccc(cc2)OCCCCOc2ccc(cc2)CCC(=O)c2cncc(n2)C(=O)N1 ; CC(C)C[C@H](NC(=O)[C@@H]1Cc2ccc(cc2)OCCCCOc2ccc(cc2)CCC(=O)c2ccc([nH]2)C(=O)N1)B(O)O]

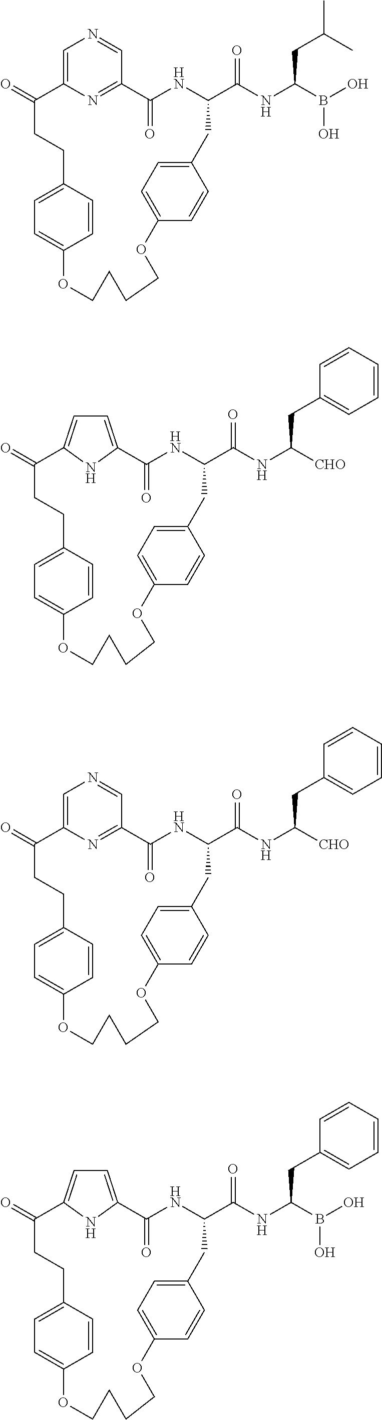 [SMILES: CC(C)C[C@H](NC(=O)[C@@H]1Cc2ccc(cc2)OCCCCOc2ccc(cc2)CCC(=O)c2cncc(n2)C(=O)N1)B(O)O ; O=C[C@H](Cc1ccccc1)NC(=O)[C@@H]1Cc2ccc(cc2)OCCCCOc2ccc(cc2)CCC(=O)c2ccc([nH]2)C(=O)N1 ; O=C[C@H](Cc1ccccc1)NC(=O)[C@@H]1Cc2ccc(cc2)OCCCCOc2ccc(cc2)CCC(=O)c2cncc(n2)C(=O)N1 ; O=C1CCc2ccc(cc2)OCCCCOc2ccc(cc2)C[C@@H](C(=O)N[C@@H](Cc2ccccc2)B(O)O)NC(=O)c2ccc1[nH]2]

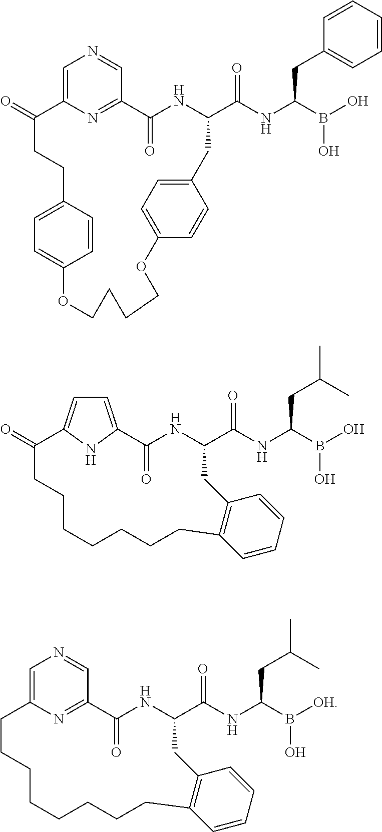 [SMILES: O=C1CCc2ccc(cc2)OCCCCOc2ccc(cc2)C[C@@H](C(=O)N[C@@H](Cc2ccccc2)B(O)O)NC(=O)c2cncc1n2 ; CC(C)C[C@H](NC(=O)[C@@H]1Cc2ccccc2CCCCCCCC(=O)c2ccc([nH]2)C(=O)N1)B(O)O ; CC(C)C[C@H](NC(=O)[C@@H]1Cc2ccccc2CCCCCCCCc2cncc(n2)C(=O)N1)B(O)O]

25. The compound according to claim 1 for use as a protease inhibitor.

26.-29. (canceled)

30. A compound of one of the following formulas and/or a pharmaceutically acceptable salt, solvate, tautomer, hydrate or prodrug derivative thereof:

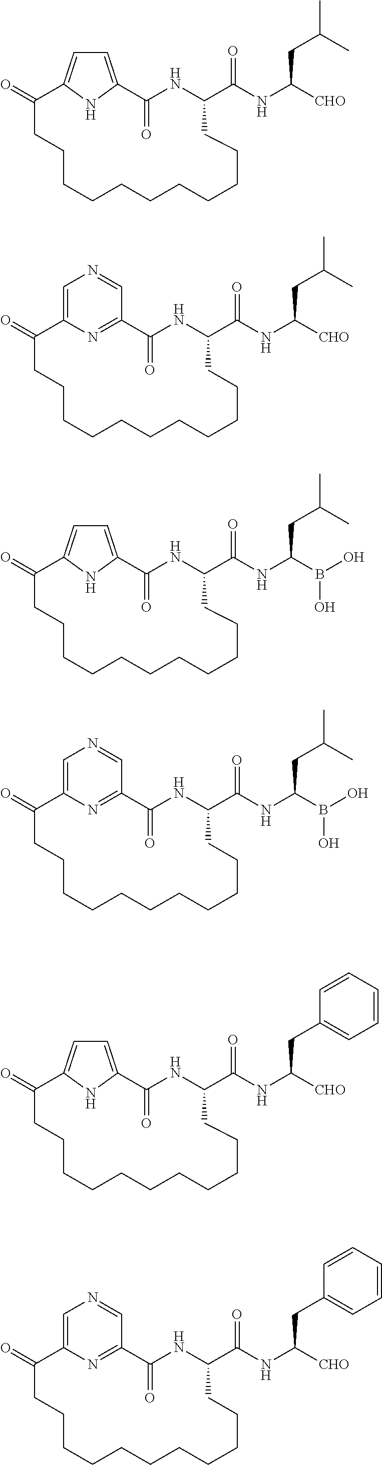 [SMILES: CC(C)C[C@@H](C=O)NC(=O)[C@@H]1CCCCCCCCCCCC(=O)c2ccc([nH]2)C(=O)N1 ; CC(C)C[C@@H](C=O)NC(=O)[C@@H]1CCCCCCCCCCCC(=O)c2cncc(n2)C(=O)N1 ; CC(C)C[C@H](NC(=O)[C@@H]1CCCCCCCCCCCC(=O)c2ccc([nH]2)C(=O)N1)B(O)O ; CC(C)C[C@H](NC(=O)[C@@H]1CCCCCCCCCCCC(=O)c2cncc(n2)C(=O)N1)B(O)O ; O=C[C@H](Cc1ccccc1)NC(=O)[C@@H]1CCCCCCCCCCCC(=O)c2ccc([nH]2)C(=O)N1 ; O=C[C@H](Cc1ccccc1)NC(=O)[C@@H]1CCCCCCCCCCCC(=O)c2cncc(n2)C(=O)N1]

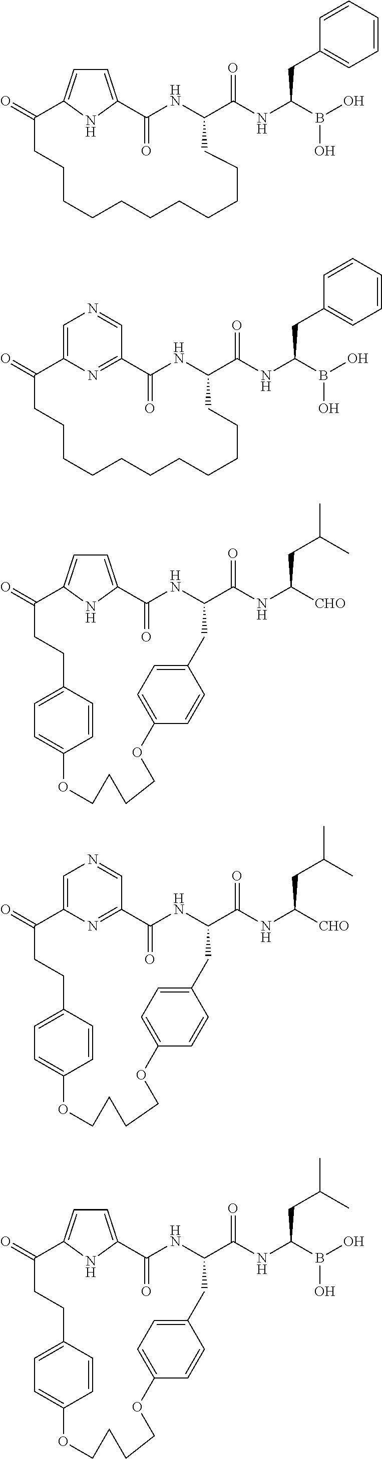 [SMILES: O=C1CCCCCCCCCCC[C@@H](C(=O)N[C@@H](Cc2ccccc2)B(O)O)NC(=O)c2ccc1[nH]2 ; O=C1CCCCCCCCCCC[C@@H](C(=O)N[C@@H](Cc2ccccc2)B(O)O)NC(=O)c2cncc1n2 ; CC(C)C[C@@H](C=O)NC(=O)[C@@H]1Cc2ccc(cc2)OCCCCOc2ccc(cc2)CCC(=O)c2ccc([nH]2)C(=O)N1 ; CC(C)C[C@@H](C=O)NC(=O)[C@@H]1Cc2ccc(cc2)OCCCCOc2ccc(cc2)CCC(=O)c2cncc(n2)C(=O)N1 ; CC(C)C[C@H](NC(=O)[C@@H]1Cc2ccc(cc2)OCCCCOc2ccc(cc2)CCC(=O)c2ccc([nH]2)C(=O)N1)B(O)O]

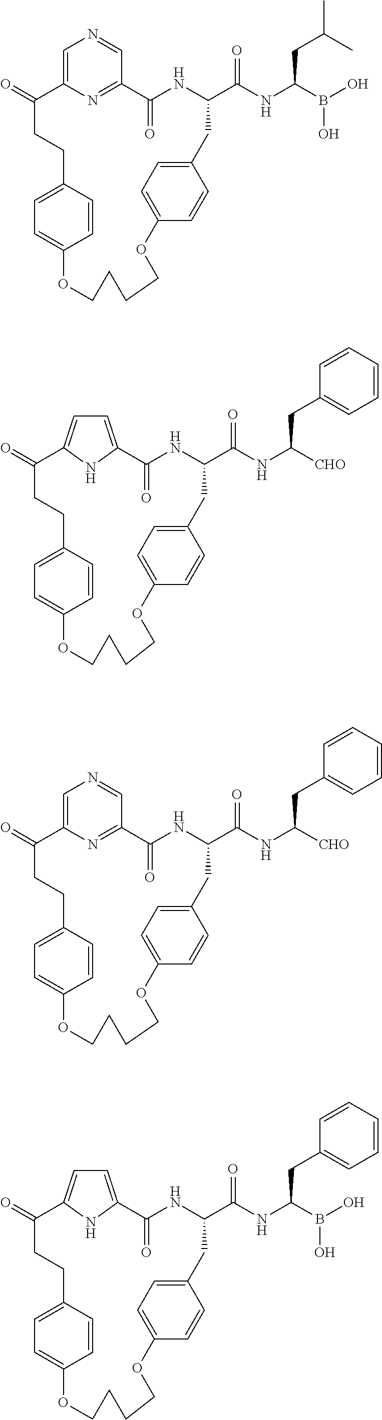 [SMILES: CC(C)C[C@H](NC(=O)[C@@H]1Cc2ccc(cc2)OCCCCOc2ccc(cc2)CCC(=O)c2cncc(n2)C(=O)N1)B(O)O ; O=C[C@H](Cc1ccccc1)NC(=O)[C@@H]1Cc2ccc(cc2)OCCCCOc2ccc(cc2)CCC(=O)c2ccc([nH]2)C(=O)N1 ; O=C[C@H](Cc1ccccc1)NC(=O)[C@@H]1Cc2ccc(cc2)OCCCCOc2ccc(cc2)CCC(=O)c2cncc(n2)C(=O)N1 ; O=C1CCc2ccc(cc2)OCCCCOc2ccc(cc2)C[C@@H](C(=O)N[C@@H](Cc2ccccc2)B(O)O)NC(=O)c2ccc1[nH]2]

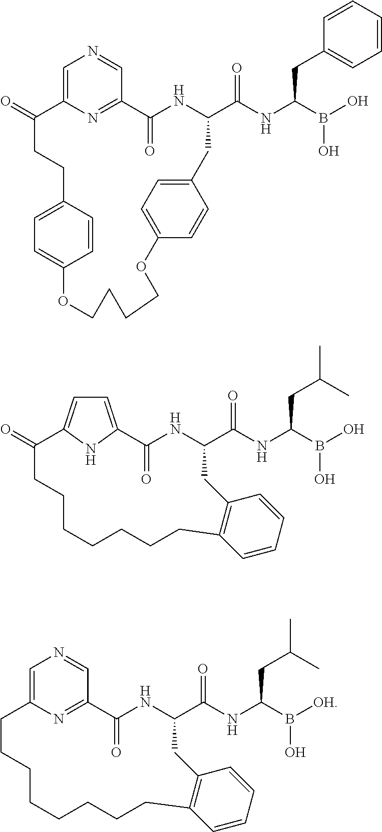 [SMILES: O=C1CCc2ccc(cc2)OCCCCOc2ccc(cc2)C[C@@H](C(=O)N[C@@H](Cc2ccccc2)B(O)O)NC(=O)c2cncc1n2 ; CC(C)C[C@H](NC(=O)[C@@H]1Cc2ccccc2CCCCCCCC(=O)c2ccc([nH]2)C(=O)N1)B(O)O ; CC(C)C[C@H](NC(=O)[C@@H]1Cc2ccccc2CCCCCCCCc2cncc(n2)C(=O)N1)B(O)O]

31. A method for inhibiting a protease, the method comprising exposing the protease to a compound according to claim 1.

32. (canceled)

33. A medicament comprising the compound of claim 1.

34. A pharmaceutical composition comprising a therapeutically effective amount of one or more compounds according to claim 1.

35. A method of preventing and/or treating a disease, condition or state in a subject associated with dysregulation of protease activity and/or dysregulation of proteosome activity, the method comprising administering to the subject a therapeutically effective dose of one or more compounds according to claim 1.

36-37. (canceled)

38. The method according to claim 35, wherein the disease, condition or state is associated with dysregulation of activity of a calpain and comprises one or more of an ocular disorder, a cataract, an optic neuropathy, ischemic optic neuropathy, diabetic neuropathy, diabetic macular oedema, glaucoma, macular degeneration, retinal ischaemia, retinal damage, retinal detachment, and presbyopia.

39. The method according to claim 38, wherein the disease, condition or state comprises one or more of an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy, and a multiple myeloma.

40. A method of preventing and/or treating an ocular disorder, a cataract, an optic neuropathy, ischemic optic neuropathy, diabetic neuropathy, diabetic macular oedema, glaucoma, macular degeneration, retinal ischaemia, retinal damage, retinal detachment, presbyopia, an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy and a multiple myeloma, the method comprising administering to the subject a therapeutically effective dose of a compound of Formula I and/or a pharmaceutically acceptable salt, solvate, tautomer, hydrate or prodrug derivative thereof: 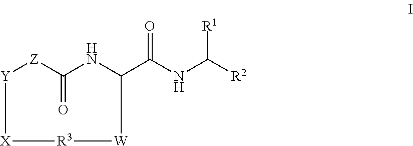
wherein:
R¹ is alkyl, aryl, heteroalkyl, or a side chain of a natural or non-natural alpha amino acid;
R² is CH(═O), CHR⁴OH, C(═O)R⁴, C(═O)C(═O)NHR⁴, CHR⁴NHR⁴, B(OH)₂, or a heterocycle, and R⁴ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
Z is a heterocycle or a heteroaryl;
Y is C(═O), C(═S), C(═NR⁵), CH₂, CHR⁵, and R⁵ is H, optionally substituted alkyl, optionally substituted heteroalkyl, optionally substituted aryl, optionally substituted heteroaryl, optionally substituted aryloxy, optionally substituted heteroaryloxy, optionally substituted arylalkoxy or optionally substituted heteroarylalkoxy;
W is no atom, alkyl, heteroalkyl, heterocycle, aryl, or heteroaryl;
R³ is alkyl, heteroalkyl, heterocycle, aryl, heteroaryl; and
X is no atom, alkyl, heteroalkyl, heterocycle, aryl, or heteroaryl.

41.-45. (canceled)

46. A method of identifying a therapeutic agent for preventing and/or treating a disease, condition or state associated with dysregulation of protease activity and/or dysregulation of proteosome activity, the method comprising:
(i) providing a P1-P3 or a P2-P4 macrocyclic peptidomimetic compound;
(ii) determining the ability of the P1-P3 or the P2-P4 macrocyclic peptidomimetic compound to prevent and/or treat a disease, condition or state associated with dysregulation of protease activity and/or dysregulation of proteosome activity; and
(iii) identifying the P1-P3 or the P2-P4 macrocyclic peptidomimetic compound as a therapeutic agent for preventing and/or treating a disease, condition or state associated with dysregulation of protease activity and/or dysregulation of proteosome activity.

47-55. (canceled)

56. The compound according to claim 2 for use as a protease inhibitor.

57. A method for inhibiting a protease, the method comprising exposing the protease to a compound according to claim 2.

58. A medicament comprising the compound according to claim 2.

59. A pharmaceutical composition comprising a therapeutically effective amount of one or more compounds according to claim 2.

60. A method of preventing and/or treating a disease, condition or state in a subject associated with dysregulation of protease activity and/or dysregulation of proteosome activity, the method comprising administering to the subject a therapeutically effective dose of one or more compounds according to claim 2.

61. The method according to claim 60, wherein the disease, condition or state is associated with dysregulation of activity of a calpain and comprises one or more of an ocular disorder, a cataract, an optic neuropathy, ischemic optic neuropathy, diabetic neuropathy, diabetic macular oedema, glaucoma, macular degeneration, retinal ischaemia, retinal damage, retinal detachment, and presbyopia.

62. The method according to claim 61, wherein the disease, condition or state comprises one or more of an inflammatory disease, condition or state, an immunological disease, condition or state, rheumatoid arthritis, pancreatitis, multiple sclerosis, an inflammation of the gastro-intestinal system, ulcerative or non-ulcerative colitis, Crohn's disease, a cardiovascular disease, condition or state, a cerebrovascular disease, condition or state, arterial hypertension, septic shock, cardiac or cerebral infarctions of ischemic or hemorrhagic origin, ischemia, a disorder linked to platelet aggregation; a disorders of the central or peripheral nervous system, a neurodegenerative disease, cerebral or spinal cord trauma, sub-arachnoid haemorrhage, epilepsy, ageing, senile dementia, Alzheimer's disease, Huntington's chorea, Parkinson's disease, a peripheral neuropathy; osteoporosis, a muscular dystrophy, cachexia, a proliferative disease, atherosclerosis, recurrence of stenosis, loss of hearing, organ transplant, an auto-immune disease, condition or state, a viral disease, lupus, AIDS, a parasitic or a viral infection, diabetes and its complications, multiple sclerosis; cancer, solid cancer, a blood-borne cancer, a malignancy, and a multiple myeloma.
